(19) United States
(12) Patent Application Publication
(10) Pub. No.: US 2023/0102101 A1
(43) Pub. Date: Mar. 30, 2023

(54) HUMAN-LIKE HEAVY CHAIN ANTIBODY VARIABLE DOMAIN (VHH) DISPLAY LIBRARIES

(71) Applicant: Merck Sharp & Dohme LLC, Rahway, NJ (US)

(72) Inventors: Lei Chen, Westborough, MA (US); Ming-Tang Chen, Dover, MA (US); Chung-Ming Hsieh, Newton, MA (US); Alexander Mario Sevy, San Diego, CA (US)

(73) Assignee: Merck Sharp & Dohme LLC, Rahway, NJ (US)

(21) Appl. No.: 17/801,471

(22) PCT Filed: Mar. 1, 2021
(86) PCT No.: PCT/US2021/020180

§ 371 Date: Aug. 22, 2022

Related U.S. Application Data
(60) Provisional application No. 62/985,537, filed on Mar. 5, 2020.

(51) Int. Cl. C07K 16/00; C12N 15/10

(52) U.S. Cl. CPC C07K 16/005; C12N 15/1037; C07K 2317/24; C07K 2317/565; C07K 2317/569; C07K 2317/92

(57) ABSTRACT

Heavy chain antibody variable domain (V_{H}H) display libraries are described comprising human-like V_{H}H comprising three synthetically generated complementarity determining region (CDR) areas in which the amino acids at each of positions 44 and 45 or positions 37, 44, 45, and 47 comprise the amino acid at the corresponding position of a Camelid V_{H}H, wherein the amino acid positions are according to Kabat numbering Human-like V_{H}Hs identified using these libraries may be useful for the manufacture of therapeutics for treating diseases and disorders.

### BACKGROUND OF THE INVENTION

### (1) Field of the Invention

The present invention relates to heavy chain antibody variable domain (V_{H}H) display libraries comprising human-like V_{H}H comprising three synthetically generated complementarity determining region (CDR) areas in which the amino acids at each of positions 44 and 45 or positions 37, 44, 45, and 47 comprise the amino acid at the corresponding position of a Camelid V_{H}H, wherein the amino acid positions are according to Kabat numbering.

### (2) Description of Related Art

Monoclonal antibody therapeutics have seen tremendous growth in recent years, with the number of approved antibody therapeutics nearly tripling between 2010 and 2019 (Kaplon et al. MAbs 12, e1703531 (2020)). In addition to the traditional full-length IgG format, there has been sustained interest in developing single-domain antibody (sdAb) therapeutics as well. Such single-domain formats include human heavy-chain only antibodies (Rouet et al., J. Biol. Chem. 290, 11905-11917 (2015); To et al., J. Biol. Chem. 280, 41395-41403 (2005)), camelid V_{H}H (Hamers-Casterman et al., Nature 363, 446-448 (1993); Muyldermans, Annu. Rev. Biochem. 82, 775-797 (2013)) and shark VNAR (Ubah et al., Biochem. Soc. Trans. 46, 1559-1565 (2018); Wesolowski et al., Med. Microbiol. Immunol. 198, 157-174 (2009)) as well as engineered formats not naturally produced by any organism (Saerens et al., Curr. Opin. Pharmacol. 8, 600-608 (2008); Vazquez-Lombardi et al., Drug Discov. Today 20, 1271-1283 (2015)). Among these a format of particular interest is camelid V_{H}H, which has the following advantages: 1) small size, 2) ease of production, 3) sequence similarity to human antibodies, minimizing immunogenicity, and 4) modularity that allows domains to be combined to form multi-specifics. Recently V_{H}H have been developed to combat infectious diseases (Sarker et al., Gastroenterol. 145, 740-748.e8 (2013); Laursen et al., Science 362, 598-602 (2018)) and the first V_{H}H was caplacizumab for acquired thrombotic thrombocytopenic purpura (aTTP) approved by the FDA for human use in 2019 (Morrison, Nat. Rev. Drug Discov. 18, 485-487 (2019)) with multiple V_{H}H currently in clinical trials (Kaplon et al., Op. Cit; Iezzi et al., Frontiers in Immunology (2018). doi:10.3389/fimmu.2018.002731).

Currently the most common method for generating V_{H}H is by animal immunization with the antigen of interest and isolation of antigen-specific B cells. This approach can be challenging, given that animal immunization is expensive, time-consuming, and not amenable to all antigen types (i.e. antigens unstable at 37° C. for prolonged periods of time). In addition, there is no control over human likeness or developability of the lead molecules, as well as the fact that not all antibodies recovered from an animal are V_{H}H.

### BRIEF SUMMARY OF THE INVENTION

To address the above limitations to generating V_{H}H of therapeutic value, the present invention provides a synthetic yeast or bacteriophage display platform for in vitro selection of antigen-specific human-like V_{H}Hs which may be used for preparing therapeutics for treatment of diseases and disorders. In this format, human-like V_{H}H genes are synthesized and cloned into a display vector adapted for use in yeast display or bacteriophage display wherein the V_{H}H are expressed and displayed on the surface of the yeast or bacteriophage, which can then be separated from each other based on their antigen binding characteristics. Specifically, the human-like V_{H}Hs comprise synthetically generated complementarity determining regions (CDRs) in a V_{H}H in which frameworks 1, 2, and 3 of the V_{H}H are humanized and framework 2 is humanized but wherein the amino acids at positions 44 and 45 or 37, 44, 45, and 47 have the amino acids in the corresponding positions of a V_{H}H of a Camelid heavy chain antibody.

The human-like V_{H}H libraries used in the present invention confer several advantages over the V_{H}H libraries currently being used in the art: (i) the human-like V_{H}H libraries are based on structural and sequence data to introduce diversity in the CDR1+2 loops only where it may contribute to antigen binding, thereby keeping amino acid sequences close to germline to minimize developability concerns; and (ii) to eliminate the need to humanize V_{H}H later on as is required using the current V_{H}H libraries in the art, the human-like V_{H}H libraries comprise a human-like framework 2 comprising the amino acids at positions 44 and 45 that are the same as the amino acids at the corresponding positions in a Camelid V_{H}H or the amino acids at positions 37, 44, 45, and 47 that are the same as the amino acids at the corresponding positions in a Camelid V_{H}H.

The V_{H}H libraries for use in the yeast display platform may use a switchable display/secretion system to enable rapid characterization of lead molecules as describes in Shaheen et al., PLoS One 8, e70190 (2013); U.S. Pat. Nos. 9,365,846; and, 10,106,598. The human-like V_{H}Hs identified using these libraries may be useful for the manufacture of therapeutics for treating diseases and disorders.

The present invention provides a nucleic acid molecule library comprising a plurality of nucleic acid molecules, each nucleic acid molecule encoding a human-like VHH comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering.

The present invention further provides a library of human-like VHHs, each VHH comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering.

The present invention further provides a human-like VHH comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering.

The present invention further provides a vector comprising a nucleic acid molecule encoding the human-like VHH of any one of the foregoing embodiments. The present invention further provides a host cell comprising the vector. In a further embodiments of the host cell, the host cell further includes a vector that encodes an Fc region of an immunoglobulin fused to a cell surface anchoring moiety that enables the Fc fusion protein to be displayed on the outer surface of the host cell. In a further embodiments of the host cell, the host cell is a yeast or filamentous fungus. In a further embodiments of the host cell, the host cell is a Saccharomyces cerevisiae or Pichia pastoris strain. The present invention further provides a library of host cells comprising the library of nucleic acid molecules that encode the human-like VHH disclosed herein.

The present invention further provides a bacteriophage comprising a nucleic acid molecule encoding the human-like VHH of any one embodiments of the nucleic acid molecules fused to a bacteriophage coat protein or to a first peptide that is capable of binding to a second peptide fused to a bacteriophage coat protein that is displayed on the outer surface of the bacteriophage and which is encoded by a second nucleic acid molecule. The present invention further provides a library of bacteriophage comprising the library of nucleic acid molecules that encode the human-like VHH disclosed herein.

The present invention further provides a display system for displaying a human-like heavy chain antibody variable domain (VHH) on the outer surface of a host cell comprising

(a) a plurality of first expression vectors, each first expression vector comprising a nucleic acid molecule encoding (i) a human-like VHH fusion protein comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering, and (ii) a first Fc polypeptide;

(b) a multiplicity of second expression vectors, each second expression vector comprising a nucleic acid molecule encoding a bait polypeptide comprising a second Fc polypeptide fused to a polypeptide or peptide that enables the second Fc polypeptide to be displayed on the outer surface of a host cell, the first and second Fc polypeptides acting, when the human-like VHH fusion protein is produced in the host cell, to cause the display of the human-like VHH fusion protein via pairwise interaction between the first and second Fc polypeptides; and

(c) host cells for transforming with the plurality of first expression vectors and multiplicity of second expression vectors.

The present invention further provides a bacteriophage display system for displaying a human-like heavy chain antibody variable domain (VHH) on the outer surface of a bacteriophage, comprising a plurality of bacteriophage, each bacteriophage comprising a nucleic acid molecule encoding a fusion protein comprising

(a) comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering, and

(b) a bacteriophage coat protein or a first peptide that is capable of binding to a second peptide fused to a bacteriophage coat protein that is displayed on the outer surface of the bacteriophage and which is encoded by a second nucleic acid molecule provided by a helper bacteriophage.

The present invention further provides a method for identifying a human-like VHH that binds a target of interest, the method comprising

(a) providing a plurality of transformed host cells comprising
(i) a plurality of first expression vectors, each first expression vector comprising a nucleic acid molecule encoding a human-like VHH fusion protein comprising
   (aa) comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering, and
   (bb) a first Fc polypeptide; and
(ii) a multiplicity of second expression vectors, each second expression vector comprising a nucleic acid molecule encoding a bait polypeptide comprising a second Fc polypeptide fused to a polypeptide or peptide that enables the second Fc polypeptide to be displayed on the outer surface of a host cell, the first and second Fc polypeptides acting, when the human-like VHH fusion protein is produced in the host cell, to cause the display of the human-like VHH fusion protein via pairwise interaction between the first and second Fc polypeptides;

(b) cultivating the transformed host cells under conditions to induce expression of the human-like VHH fusion proteins and the bait polypeptide to produce induced host cells in which the bait polypeptide is displayed on the outer surface of the transformed host cells and the human-like VHH fusion protein is in a pairwise interaction with the bait polypeptide;

(c) contacting the induced host cells with the target of interest conjugated to a detection moiety; and

(d) detecting the detection moiety and selecting the host cells that express the human-like VHH fusion protein that binds the target of interest.

In a further embodiment of the method, the host cell is a yeast or filamentous fungus. In a further embodiment of the method, the host cell is a Saccharomyces cerevisiae or Pichia pastoris strain.

The present invention further provides a method for identifying a human-like VHH that binds a target of interest, the method comprising

(a) providing a recombinant bacteriophage library, each bacteriophage comprising a nucleic acid molecule encoding a fusion protein comprising a bacteriophage coat protein fused to a human-like VHH comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering, and displaying the fusion protein on the outer surface thereof

(b) contacting the recombinant bacteriophage library with the target of interest immobilized on a solid support;

(c) removing the recombinant bacteriophage in the library that do not bind the target of interest and eluting the recombinant bacteriophage bound to the target of interest to provide recombinant bacteriophage that bind the target of interest;

(d) repeating steps (b) and (c) one to three times to provide a population of recombinant bacteriophage enriched for recombinant bacteriophage that bind the target of interest; and

(d) determining the amino acid sequence of the human-like VHH to provide the human-like VHH that binds the target of interest.

In each of the foregoing inventions and embodiments, the human VH framework further includes substitution of each of the amino acids at positions 37 and 47 with the amino acid at corresponding positions 37 and 47 of the Camelid VHH framework, wherein the amino acid positions are according to Kabat numbering.

In each of the foregoing inventions and embodiments, the human VH framework comprises the amino acid sequence of the human VH framework encoded by the IGHV3-23*04 gene in which the amino acids at positions 44 and 45 of the human VH framework are each substituted with the corresponding amino acid at positions 44 and 45 of the Camelid VHH framework encoded by the alpaca IGHV3S53 gene, wherein the amino acid positions are according to Kabat numbering.

In each of the foregoing inventions and embodiments, the human VH framework comprises the amino acid sequence of the human VH framework encoded by the IGHV3-23*04 gene and the amino acids at positions 37, 44, 45, and 47 of the human VH framework are each substituted with the corresponding amino acid at positions 37, 44, 45, and 47 of the Camelid VHH framework encoded by the alpaca IGHV3S53 gene, wherein the amino acid positions are according to Kabat numbering.

The present invention provides a nucleic acid molecule library comprising a plurality of nucleic acid molecules, each nucleic acid molecule encoding a human-like V_{H}H comprising three synthetically generated CDR areas in a human-like V_{H}H framework in which the amino acids at each of positions 44 and 45 of the human-like V_{H} framework correspond to the amino acids at positions 44 and 45 of a Camelid V_{H}H framework, wherein the amino acid positions are according to Kabat numbering.

The present invention further provides a library of human-like V_{H}Hs, each V_{H}H comprising three synthetically generated CDR areas in a human-like V_{H}H framework in which the amino acids at each of positions 44 and 45 of the human-like V_{H}H framework correspond to the amino acids at positions 44 and 45 of a Camelid V_{H}H framework, wherein the amino acid positions are according to Kabat numbering.

The present invention further provides a human-like V_{H}H comprising three synthetically generated CDR)areas in a human antibody heavy chain variable domain (V_{H}) framework in which the amino acids at each of positions 44 and 45 of the human V_{H} framework correspond to the amino acids at positions 44 and 45 of a Camelid V_{H}H framework, wherein the amino acid positions are according to Kabat numbering.

The present invention provides a nucleic acid molecule library comprising a plurality of nucleic acid molecules, each nucleic acid molecule encoding a human-like V_{H}H comprising three synthetically generated CDR areas in a human-like V_{H}H framework in which the amino acids at each of positions 37, 44, 45, and 47 of the human-like V_{H} framework correspond to the amino acids at positions 37, 44, 45, and 47 of a Camelid V_{H}H framework, wherein the amino acid positions are according to Kabat numbering.

The present invention further provides a library of human-like V_{H}Hs, each V_{H}H comprising three synthetically generated CDR areas in a human-like V_{H}H framework in which the amino acids at each of positions 37, 44, 45, and 47 of the human-like V_{H}H framework correspond to the amino acids at positions 37, 44, 45, and 47 of a Camelid V_{H}H framework, wherein the amino acid positions are according to Kabat numbering.

The present invention further provides a human-like V_{H}H comprising three synthetically generated CDR areas in a human V_{H} framework in which the amino acids at each of positions 37, 44, 45, and 47 of the human V_{H} framework correspond to the amino acids at positions 37, 44, 45, and 47 of a Camelid V_{H}H framework, wherein the amino acid positions are according to Kabat numbering.

In the further embodiments, the Camelid V_{H}H is encoded by the alpaca IGHV3S53 gene. In the above embodiments, the amino acid at positions 37, 44, 45, and 47 are Tyr, Gln, Arg, and Leu, respectively.

In further embodiments, the human-like V_{H}H comprises amino acids at positions 1, 27, 28, 32, 49, 58, 74, 78, 83, 84, 93, and 94 that are the same as the amino acids at the corresponding positions in a human V_{H}, or the human-like V_{H}H comprises amino acids at positions 1, 27, 28, 32, 35, 49, 58, 74, 78, 83, 84, 93, and 94 that are the same as the amino acids at the corresponding positions in a human V_{H}, or the human-like V_{H}H comprises amino acids at positions 1, 27, 28, 32, 49, 52, 58, 74, 78, 83, 84, 93, and 94 that are the same as the amino acids at the corresponding positions in a human V_{H}, wherein the amino acid positions are according to Kabat numbering. In further embodiments, the amino acid positions correspond to the V_{H} encoded by the human IGHV3-23*04 gene. In a further embodiments, frameworks 1, 3, and 4 have the same amino acid sequence as framework 1, 3, and 4 of a V_{H} encoded by the human IGHV3-23*04 gene and framework 2 has the same amino acid as a framework of a V_{H} encoded by the human IGHV3-23*04 gene except that amino acids at positions 44 and 45 or positions 37, 44, 45, and 47 are the same amino acids as the amino acids at the corresponding positions in a V_{H}H encoded by the alpaca IGHV3S53 gene except.

The present invention further provides a vector comprising a nucleic acid molecule encoding the human-like V_{H}H of any one of the foregoing embodiments. The present invention further provides a host cell comprising the vector. In a further embodiments of the host cell, the host cell further includes a vector that encodes an Fc region of an immunoglobulin fused to a cell surface anchoring moiety that enables the Fc fusion protein to be displayed on the outer surface of the host cell. In a further embodiments of the host cell, the host cell is a yeast or filamentous fungus. In a further embodiments of the host cell, the host cell is a Saccharomyces cerevisiae or Pichia pastoris strain. The present invention further provides a library of host cells comprising the library of nucleic acid molecules that encode the human-like V_{H}H disclosed herein.

The present invention further provides a bacteriophage comprising a nucleic acid molecule encoding the human-like V_{H}H of any one embodiments of the nucleic acid molecules fused to a bacteriophage coat protein or to a first peptide that is capable of binding to a second peptide fused to a bacteriophage coat protein that is displayed on the outer surface of the bacteriophage and which is encoded by a second nucleic acid molecule. The present invention further provides a library of bacteriophage comprising the library of nucleic acid molecules that encode the human-like V_{H}H disclosed herein.

The present invention further provides a display system for displaying a human-like V_{H}H on the outer surface of a host cell comprising (a) a plurality of first expression vectors, each first expression vector comprising a nucleic acid molecule encoding (i) a human-like V_{H}H fusion protein comprising three synthetically generated CDR areas in a human V_{H} framework in which the amino acids at each of positions 44 and 45 of the human V_{H} framework correspond to the amino acids at positions 44 and 45 of a Camelid V_{H}H framework, wherein the amino acid positions are according to Kabat numbering, and (ii) a first Fc polypeptide;

(b) a multiplicity of second expression vectors, each second expression vector comprising a nucleic acid molecule encoding a bait polypeptide comprising a second Fc polypeptide fused to a polypeptide or peptide that enables the second Fc polypeptide to be displayed on the outer surface of a host cell, the first and second Fc polypeptides acting, when the human-like V_{H}H fusion protein is produced in the host cell, to cause the display of the human-like V_{H}H fusion protein via pairwise interaction between the first and second Fc polypeptides; and

(c) host cells for transforming with the plurality of first expression vectors and multiplicity of second expression vectors.

The present invention further provides a bacteriophage display system for displaying a human-like heavy chain antibody variable domain (V_{H}H) on the outer surface of a bacteriophage, comprising a plurality of bacteriophage, each bacteriophage comprising a nucleic acid molecule encoding a fusion protein comprising

(a) comprising three synthetically generated complementarity determining region (CDR) areas in a human V_{H} framework in which the amino acids at each of positions 44 and 45 of the human V_{H} framework correspond to the amino acids at positions of a Camelid V_{H}H framework, wherein the amino acid positions are according to Kabat numbering, and

(b) a bacteriophage coat protein or a first peptide that is capable of binding to a second peptide fused to a bacteriophage coat protein that is displayed on the outer surface of the bacteriophage and which is encoded by a second nucleic acid molecule provided by a helper bacteriophage.

The present invention further provides a method for identifying a human-like V_{H}H that binds a target of interest, the method comprising

(a) providing a plurality of transformed host cells comprising
(i) a plurality of first expression vectors, each first expression vector comprising a nucleic acid molecule encoding a human-like V_{H}H fusion protein comprising
   (aa) comprising three synthetically generated CDR areas in a human antibody heavy chain variable domain (V_{H}) framework in which the amino acids at each of positions 44 and 45 of the human V_{H} framework correspond to the amino acids at positions of a Camelid V_{H}H framework, wherein the amino acid positions are according to Kabat numbering, and
   (bb) a first Fc polypeptide; and
(ii) a multiplicity of second expression vectors, each second expression vector comprising a nucleic acid molecule encoding a bait polypeptide comprising a second Fc polypeptide fused to a polypeptide or peptide that enables the second Fc polypeptide to be displayed on the outer surface of a host cell, the first and second Fc polypeptides acting, when the human-like V_{H}H fusion protein is produced in the host cell, to cause the display of the human-like V_{H}H fusion protein via pairwise interaction between the first and second Fc polypeptides;

(b) cultivating the transformed host cells under conditions to induce expression of the human-like V_{H}H fusion proteins and the bait polypeptide to produce induced host cells in which the bait polypeptide is displayed on the outer surface of the transformed host cells and the human-like V_{H}H fusion protein is in a pairwise interaction with the bait polypeptide;

(c) contacting the induced host cells with the target of interest conjugated to a detection moiety; and

(d) detecting the detection moiety and selecting the host cells that express the human-like V_{H}H fusion protein that binds the target of interest.

In a further embodiment of the method, the host cell is a yeast or filamentous fungus. In a further embodiment of the method, the host cell is a Saccharomyces cerevisiae or Pichia pastoris strain.

The present invention further provides a method for identifying a human-like V_{H}H that binds a target of interest, the method comprising

(a) providing a recombinant bacteriophage library, each bacteriophage comprising a nucleic acid molecule encoding a fusion protein comprising a bacteriophage coat protein fused to a human-like V_{H}H comprising three synthetically generated CDR areas in a human V_{H} framework in which the amino acids at each of positions 44 and 45 of the human V_{H} framework correspond to the amino acids at positions of a Camelid V_{H}H framework, wherein the amino acid positions are according to Kabat numbering, and displaying the fusion protein on the outer surface thereof

(b) contacting the recombinant bacteriophage library with the target of interest immobilized on a solid support;

(c) removing the recombinant bacteriophage in the library that do not bind the target of interest and eluting the recombinant bacteriophage bound to the target of interest to provide recombinant bacteriophage that bind the target of interest;

(d) repeating steps (b) and (c) one to three times to provide a population of recombinant bacteriophage enriched for recombinant bacteriophage that bind the target of interest; and

(d) determining the amino acid sequence of the human-like V_{H}H to provide the human-like V_{H}H that binds the target of interest.

In each of the foregoing inventions and embodiments, the human-like V_{H} framework further includes amino acids at positions 37 and 47 that correspond to the amino acids at positions of the Camelid V_{H}H framework, wherein the amino acid positions are according to Kabat numbering.

In each of the foregoing inventions and embodiments, the human-like V_{H} framework comprises the amino acid sequence of the human V_{H} framework encoded by the IGHV3-23*04 gene in which the amino acids at positions 44 and 45 of the human V_{H} framework are each substituted with the corresponding amino acid at positions 44 and 45 of the Camelid V_{H}H framework encoded by the alpaca IGHV3S53 gene, wherein the amino acid positions are according to Kabat numbering.

In each of the foregoing inventions and embodiments, the human V_{H} framework comprises the amino acid sequence of the human V_{H} framework encoded by the IGHV3-23*04 gene and the amino acids at positions 37, 44, 45, and 47 of the human V_{H} framework are each substituted with the corresponding amino acid at positions 37, 44, 45, and 47 of the Camelid V_{H}H framework encoded by the alpaca IGHV3S53 gene, wherein the amino acid positions are according to Kabat numbering.

The human V_{H} framework and Camelid V_{H}H framework each comprises four frameworks and three CDRs in the following sequence: (framework 1)-(CDR1)-(framework 2)-(CDR2)-(framework 3)-(CDR3)-(framework 4).

Thus, in each of the foregoing inventions and embodiments, the amino acids at position 37, 44, 45, and/or 47 of the human-like V_{H}H are Tyr, Gln, Arg, and/or Leu, respectively and the remainder of the amino acids in the frameworks are the same as the amino acids in the corresponding positions of a human V_{H}.

In particular embodiments, the human-like V_{H}H framework comprises an amino acid sequence that is the same as the corresponding amino acid sequence of the human V_{H} framework except that positions 44 and 45 of the framework are Gln and Arg, respectively, which in certain embodiments, the human V_{H} framework is encoded by the IGHV3-23*04 gene. In particular embodiments, human V_{H} frameworks 1, 3, and 4 may comprise 1, 2, 3,4, or 5 amino acid substitutions.

In particular embodiments, the human-like V_{H}H framework comprises an amino acid sequence that is the same as the corresponding amino acid sequence of the human V_{H} framework except that positions 37, 44, 45, and 47 of the framework are Tyr, Gln, Arg, and Leu, respectively, which in certain embodiments, the human V_{H} framework is encoded by the IGHV3-23*04 gene. In particular embodiments, human V_{H} frameworks 1, 3, and 4 may comprise 1, 2, 3,4, or 5 amino acid substitutions.

In particular embodiments, the human-like V_{H}H framework 2 comprises an amino acid sequence that is the same as the corresponding amino acid sequence of the human V_{H} framework 2 except that positions 37, 44, 45, and 47 of the framework 2 are Tyr, Gln, Arg, and Leu, respectively, which in certain embodiments, the human V_{H} framework 2 is encoded by the IGHV3-23*04 gene. In particular embodiments, human V_{H} frameworks 1, 3, and 4 may comprise 1, 2, 3,4, or 5 amino acid substitutions. In further embodiments, human V_{H} frameworks 1, 3, and 4 comprise the amino acid sequences native to the human V_{H} framework 1, 3, and 4 of the human V_{H} framework. In particular embodiments, human V_{H} frameworks 1, 3, and 4 may comprise 1, 2, 3,4, or 5 amino acid substitutions.

In specific embodiments of each of the foregoing inventions and embodiments, the human-like V_{H}H comprising the library may comprise the amino acid sequence of one or more of the following human-like V_{H}H amino acid sequences
EVQLVESGGGLVQPGGSLRLSCAASGFTFSXYXMSWYRQAPGKQRELVSAIXSGGXTY YADSVKGRFTISRDNSKNTLYLQMNSLRAEDTAVYYCARXXXXXXXXXXXXXXXFDX WGQGTLVTVSS (SEQ ID NO: 1)), wherein each occurrence of X is independently any amino acid except C;
EVQLLESGGGLVQPGGSLRLSCAASGFTFXXYAMXWVRQAPGKQREWVSXISXXGXX TYYADSVKGRFTISRDNSKNTLYLQMNSLRAEDTAVYYCARXXXXXXXXXXXXXXXF DXWGQGTLVTVSS (SEQ ID NO: 2), wherein each occurrence of X is independently any amino acid except C;
EVQLLESGGGLVQPGGSLRLSCAASGFTFXXYAMXWVRQAPGKQREWVSXISXXGXX TYYADSVKGRFTISRDNSKNTLYLQMNSLRAEDTAVYYCARXXXXXXXXXXXXXXXF DXWGQGTLVTVSS (SEQ ID NO: 3), wherein each occurrence of X is independently any amino acid except C; or
EVQLLESGGGLVQPGGSLRLSCAASGFTFXXYAMXWYRQAPGKQRELVSXISXXGXXT YYADSVKGRFTISRDNSKNTLYLQMNSLRAEDTAVYYCARXXXXXXXXXXXXXXXFD XWGQGTLVTVSS (SEQ ID NO: 4), wherein each occurrence of X is independently any amino acid except C.

### BRIEF DESCRIPTION OF THE DRAWINGS

FIG. 1A shows by illustration the identification and filtration of V_{H}H-antigen complex structures in the Protein DataBank analyzed using the Rosetta modeling software (Alford, R. F. et al. The Rosetta All-Atom Energy Function for Macromolecular Modeling and Design. J. Chem. Theory Comput. 13, 3031-3048 (2017)).

FIG. 1B shows the average contribution to total binding energy by antibody region for each V_{H}H-antigen complex. The total binding energy was calculated and the percentage total binding energy was calculated per antibody region, between frameworks (FR) and CDR loops. Bars show mean±SD.

FIG. 1C shows the average per-residue binding energy calculated for each V_{H}H-antigen complex for residues in the CDRH1. Y-axis shows the average per-residue binding energy in Rosetta Energy Units (REU). Lower values indicate a stronger binding interaction. X-axis shows the residue number in Kabat numbering.

FIG. 1D shows the average per-residue binding energy calculated for each VHH-antigen complex for residues in the CDRH2. Y-axis shows the average per-residue binding energy in Rosetta Energy Units (REU). Lower values indicate a stronger binding interaction. X-axis shows the residue number in Kabat numbering.

FIG. 2A-2E show the results of next-generation sequencing (NGS) analysis of alpaca and camel V_{H}H repertoires.

FIG. 2A shows a heatmap that shows germline gene usage from an alpaca sequencing dataset. Sequences were aligned to the Vicugna pacos IGHV and IGHJ reference genes from IMGT (Lo, B. K. C. & Lefranc, M.-P. IMGT, The International ImMunoGeneTics Information System®, Antib. Eng. 33, 27-50 (2004)).

FIG. 2B shows CDRH1 (panels B, D) and CDRH2 (panels C, E) amino acid profiles from IGHV3S53-encoded sequences in alpaca (panels B, C) or camel (panels D, E) repertoires. Shown below the panels is Kabat numbering for the CDRH1 and CDRH2 and below are shown IGHV3S53 germline CDRH1 sequence GSIFSINA (SEQ ID NO. 36) and CDRH2 sequence ITSGGST (SEQ ID NO: 37). Sequence logos were created using WebLogo (Crooks, G. E. WebLogo: A Sequence Logo Generator. Genome Res. 14, 1188-1190 (2004)). Amino acids are shaded by chemical properties.

FIG. 3 shows the strategy for the partial humanization of gene IGHV3S53 encoding V_{H}H for construction of the libraries. Shown is the alignment of amino acids 1-98 (SEQ ID NO: 8) of the V_{H} encoded by human gene IGHV3-23*04 (SEQ ID NO: 6), the closest human homolog to amino acids 1-97 (SEQ ID NO: 7) of the V_{H}H encoded by alpaca IGHV3S53 gene (SEQ ID NO: 5). Amino acid differences are indicated with a vertical line. All positions of difference in the alpaca IGHV3S53 sequence were reverted to the human amino acid to provide the partially humanized IGHV3S53 sequence for use in the library, except for those indicated by asterisks, which designates hallmark amino acids in the alpaca amino acid sequence that were maintained to provide V_{H}H stability. Two partially human-like frameworks were created, one maintaining four amino acids from the alpaca gene (YQRL at positions 37, 44, 45, and 47, respectively) and one maintaining two amino acids (QR at positions 44 and 45, respectively).

FIG. 4A-4B show results of an anti-mPD-1 V_{H}H campaign using the five libraries described herein (Alp_LowDiv, Hum_LowDiv. Alp_HighDiv, Hum_HighDiv, Kruse)

FIG. 4A shows flow cytometry plots of output after four rounds of FACS selection. The top row shows the libraries incubated with no antigen (only secondary detection reagents) and the bottom row shows the libraries with the addition of 50 nM mPD-1. The X-axis shows antigen binding, as detected by neutravidin-linked R-PE fluorophore, and the Y-axis shows antibody expression, as detected by an anti-HA tag monoclonal antibody conjugated to AlexaFluor 647.

FIG. 4B shows results of NGS of the library outputs. Each library was sequenced on an Illumina MiSeq 2×250. See Methods for details on read filtering.

FIG. 4C shows binding affinity of recombinant V_{H}H measured by Biolayer Interferometry (BLI).

FIG. 4D shows blocking of the PD-1-PD-L1 interaction was measured in vitro using BLI. Y-axis shows % percent blocking, where a non-blocking antibody would be 0 and a fully blocking antibody 100.

FIG. 5A-5D show results of the peptide campaign for four libraries.

FIG. 5A shows flow cytometry plots of output after four rounds of FACS selection of the anti-peptide libraries. The top row shows the libraries incubated with no peptide (only secondary detection reagents) and the bottom row shows the libraries with the addition of 10 nM peptide. The X-axis shows binding to the peptide, as detected by streptavidin-linked R-PE fluorophore, and the Y-axis shows recombinant V_{H}H expression, as detected by an anti-HA tag monoclonal antibody conjugated to AlexaFluor 647. Library Alp_LowDiv was excluded as it did not enrich peptide-specific binders over reagent binders after two rounds of selection.

FIG. 5B shows results of NGS of the anti-peptide library outputs. Each library was sequenced on an Illumina MiSeq 2×250. See Methods for details on read filtering.

FIG. 5C shows epitope mapping data for the anti-peptide libraries. Library output after four rounds of FACS selection were incubated with one of seven biotinylated peptides, and binding was detected by a neutravidin-PE secondary. A no peptide (no Ag) control was added to measure background. Mean fluorescence intensity in the PE channel is plotted on the Y-axis.

FIG. 5D shows binding affinity of recombinant V_{H}H to the peptide measured by BLI.

FIG. 6A shows results of flow cytometry plots of output after four rounds of FACS selection for an anti-GPCR campaign using the five libraries described herein ((Alp_LowDiv, Hum_LowDiv. Alp_HighDiv, Hum_HighDiv, Kruse). The top row shows the libraries incubated with no antigen (only secondary detection reagents) and the bottom row shows the libraries with the addition of 50 nM GPCR antigen. The X-axis shows antigen binding, as detected by streptavidin-linked R-PE fluorophore, and the Y-axis shows antibody expression, as detected by an anti-HA tag monoclonal antibody conjugated to AlexaFluor 647.

FIG. 6B shows Results of single clone colony PCR and FACS analysis. Shown are number of colonies sequenced from the output of FACS round number, number of unique CDR3s obtained from the sequenced colonies, as well as qualitative analysis of the results of single clone FACS binding (either no binding, reagent binding, or antigen-specific binding).

FIG. 7 shows melting temperatures of recombinant V_{H}H from four of the libraries disclosed herein (Alp_LowDiv, Hum_LowDiv. Hum_HighDiv, Kruse). No differences between libraries were significant (Mann-Whitney test, p=0.05 with Bonferroni correction for multiple comparisons).

FIGS. 8A and 8B show the properties of the Alp_LowDiv, Hum_LowDiv, Alp_HighDiv, and Hum_HighDiv naïve libraries from NGS. Pictured from left to right are CDRH3 length distributions (Kabat definition), amino acid sequence profiles for CDRH1 and CDRH2. Below the sequence logos is the residue numbering in Kabat format. Below the sequence logos is the residue numbering in Kabat format.

FIG. 9A and FIG. 9B show the properties of the Alp_LowDiv, Hum_LowDiv, Alp_HighDiv, and Hum_HighDiv libraries after mPD-1 selection from NGS. Pictured from left to right are CDRH3 length distributions (Kabat definition), amino acid sequence profiles for CDRH1 and CDRH2. Below the sequence logos is the residue numbering in Kabat format.

FIG. 10 shows representative plots for in vitro receptor blocking. Shown at top is a schematic of the assay. Biotinylated mPD-1 was loaded onto streptavidin sensors, sensor was dipped into either VHH or buffer was added, then mPD-L1 was associated. Trace A shows positive control (full receptor binding), trace C shows negative control (no mPD-L1 added), and trace B shows blocking activity (addition of V_{H}H first, mPD-L1 second). Clone name is shown above each trace. Representative plots are shown for V_{H}H with full blocking, partial blocking, or non-blocking activity. In several cases the response after mPD-L1 was lower than the negative control, due to the impact of V_{H}H dissociating from the biosensor — these samples were treated as 100% blocking.

FIG. 11 shows the Kabat numbering for the amino acid sequences of a representative low diversity human-like V_{H}H having Y37/Q44/R45/L47 amino acid substitutions in framework 2 (SEQ ID NO: 33) and representative high diversity human-like V_{H}H having Q44/R45 amino acid substitutions in framework 2 (SEQ ID NO:35).

FIG. 12A and FIG. 12B show properties of libraries after peptide selection from NGS. Pictured from left to right are CDRH3 length distributions (Kabat definition), amino acid sequence profiles for CDRH1 and CDRH2. Below the sequence logos is the residue numbering in Kabat format.

### DETAILED DESCRIPTION OF THE INVENTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

The term “Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including KinExA and Biacore. Specific illustrative and exemplary embodiments for measuring binding affinity are described in the following.

The term “administration” and “treatment,” as it applies to an animal, human, experimental subject, cell, tissue, organ, or biological fluid, refers to contact of an exogenous pharmaceutical, therapeutic, diagnostic agent, or composition comprising a human-like V_{H}H to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., human, rat, mouse, dog, cat, rabbit). In a preferred embodiment, the term “subjects” refers to a human.

The term “amino acid” refers to a simple organic compound containing both a carboxyl (—COOH) and an amino (—NH₂) group. Amino acids are the building blocks for proteins, polypeptides, and peptides. Amino acids occur in L-form and D-form, with the L-form in naturally occurring proteins, polypeptides, and peptides. Amino acids and their code names are set forth in the following chart.

| | Three letter | One letter |
|---|---|---|
| Amino add | code | code |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “antibody” or “immunoglobulin” as used herein refers to a glycoprotein comprising either (a) at least two heavy chains (HCs) and two light chains (LCs) inter-connected by disulfide bonds, or (b) in the case of a species of camelid antibody, at least two heavy chains (HCs) inter-connected by disulfide bonds. Each HC is comprised of a heavy chain variable region or domain (V_{H}) and a heavy chain constant region or domain. In certain naturally occurring IgG, IgD and IgA antibodies, the heavy chain constant region is comprised of three domains, C_{H}1, C_{H}2 and C_{H}3. In general, the basic antibody structural unit for antibodies is a tetramer comprising two HC/LC pairs, except for the species of camelid antibodies comprising only two HCs, in which case the structural unit is a homodimer. Each tetramer includes two identical pairs of polypeptide chains, each pair having one LC (about 25 kDa) and HC chain (about 50-70 kDa).

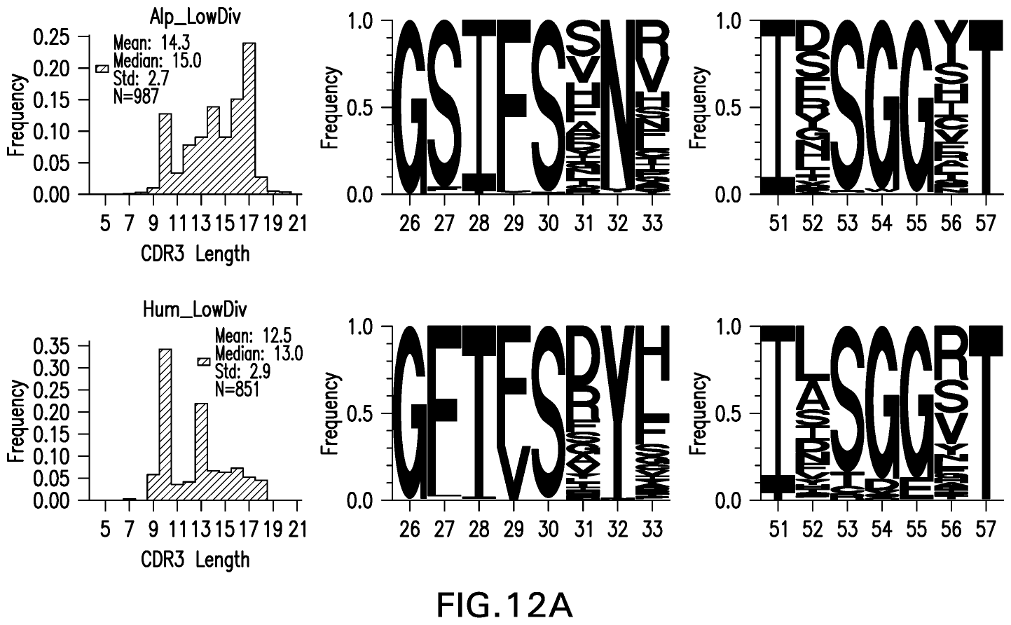

In certain naturally occurring antibodies, each light chain is comprised of an LC variable region or domain (V_{L}) and a LC constant domain. The LC constant domain is comprised of one domain, C_{L}. The human V_{H} includes seven family members: V_{H}1, V_{H}2, V_{H}3, V_{H}4, V_{H}5, V_{H}6, and V_{H}7; and the human V_{L} includes 16 family members: V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10. Each of these family members can be further divided into particular subtypes. The V_{H} and V_{L} domains can be further subdivided into regions of hypervariability, termed complementarity determining region (CDR) areas, interspersed with regions that are more conserved, termed framework regions (FR). Each V_{H} and V_{L} is composed of three CDR regions and four FR regions, arranged from amino-terminus to carboxy-terminus in the following order: FR1, CDR1, FR2, CDR2, FR3, CDR3, FR4. Numbering of the amino acids in a V_{H} or V_{H}H may be determined using Kabat numbering scheme. See Béranger, et al., Ed. Ginetoux, Correspondence between the IMGT unique numbering for C-DOMAIN, the IMGT exon numbering, the Eu and Kabat numberings: Human IGHG, Created: 17/05/2001, Version: 08/06/2016, which is accessible at www.imgt.org/IMGTScientificChart/Numbering/Hu_IGHGnber.html). For example, FIG. 11 shows the Kabat numbering for the amino acid sequences of a representative low diversity human-like V_{H}H having Y37/Q44/R45/L47 amino acid substitutions in framework 2 (SEQ ID NO: 33) and representative high diversity human-like V_{H}H having Q44/R45 amino acid substitutions in framework 2 (SEQ ID NO:35).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG₁ (Eu), which has a constant domain that begins at amino acid position 118 of the amino acid sequence of the IgG₁ described in Edelman et al., Proc. Natl. Acad. Sci. USA. 63: 78-85 (1969), and is shown for the IgG₁, IgG₂. IgG₃, and IgG₄ constant domains in Béranger, et al., Ibid.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196: 901-917 (1987); Al-Lazikani et al., J. Mol. Biol. 273: 927-948 (1997));
AbM numbering scheme is a compromise between the two used by Oxford Molecular's AbM antibody modelling software (see Karu et al, ILAR Journal 37: 132-141 (1995);
Contact numbering scheme is based on an analysis of the available complex crystal structures (See www.bioinf.org.uk: Prof. Andrew C. R. Martin's Group; Abhinandan & Martin, Mol. Immunol. 45:3832-3839 (2008).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).

The following general rules disclosed in www.bioinf.org.uk: Prof. Andrew C. R. Martin's Group and reproduced in Table 1 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 1

| Loop | Kabat | AbM | Chothia¹ | Contact² | IMGT |
|---|---|---|---|---|---|
| L1 | L24--L34 | L24--L34 | L24-L34 | L30-L36 | L27-L32 |
| L2 | L50-L56 | L50-L56 | L50-L56 | L46-L55 | L50-L51 |
| L3 | L89-L97 | L89-L97 | L89-L97 | L89-L96 | L89-L97 |
| H1 | H31-H35B | H26- | H26-H32 . . . 34 | H30-H35B | H26-H35B |
| | (Kabat Numbering)³ | H35B | | | |
| H1 | H31-H35 | H26-H35 | H26-H32 | H30-H35 | H26--H33 |
| | (Chothia Numbering) | | | | |
| H2 | H50-H65 | H50-H58 | H52-H56 | H47-H58 | H51-H56 |
| H3 | H95-H102 | H95-H102 | H95-H102 | H93-H101 | H93-H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35 A and H35B are present, the loop ends at H34

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296: 833-849 (2000); Klimka et al., British J. Cancer 83: 252-260 (2000); Rader et al., Proc. Natl. Acad. Sci. USA 95: 8910-8915 (1998); Xu et al., Immunity 13: 37-45 (2000).

The term “antigen” as used herein refers to any foreign substance which induces an immune response in the body.

The term “camelized” V_{H} refers to an ISVD in which one or more amino acid residues in the amino acid sequence of a naturally occurring V_{H} domain from a conventional four-chain antibody by one or more of the amino acid residues that occur at the corresponding position(s) in a V_{H}H domain of a heavy chain antibody. Such “camelizing” substitutions may be inserted at amino acid positions that form and/or are present at the V_{H}-V_{L} interface, and/or at the so-called Camelidae hallmark residues, as defined herein (see also for example WO9404678 and Davies and Riechmann (1994 and 1996)). Reference is made to Davies and Riechmann (FEBS 339: 285-290, 1994; Biotechnol. 13: 475-479, 1995; Prot. Eng. 9: 531-537, 1996) and Riechmann and Muyldermans (J. Immunol. Methods 231: 25-38, 1999).

The terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

The term “CDR area” refers to a CDR as defined by any one of the methods commonly used for defining CDRs and which may further include up to one amino acid N-terminal to the defined CDR or up to three amino acids C-terminal to the defined CDR.

The term “control sequences” or “regulatory sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “epitope”, as used herein, is defined in the context of a molecular interaction between a human-like V_{H}H and its corresponding “antigen” (Ag). Generally, “epitope” refers to the area or region on an Ag to which human-like V_{H}H specifically binds, i.e. the area or region in physical contact with the human-like V_{H}H. Physical contact may be defined through distance criteria (e.g. a distance cut-off of 4 Å) for atoms in the human-like V_{H}H and Ag molecules.

The epitope for a given human-like V_{H}H/Ag pair can be defined and characterized at different levels of detail using a variety of experimental and computational epitope mapping methods. The experimental methods include mutagenesis, X-ray crystallography, Nuclear Magnetic Resonance (NMR) spectroscopy and Hydrogen deuterium exchange Mass Spectrometry (HX-MS), methods that are known in the art. As each method relies on a unique principle, the description of an epitope is intimately linked to the method by which it has been determined. Thus, depending on the epitope mapping method employed, the epitope for a given Ab/Ag pair will be described differently.

The epitope for a given human-like V_{H}H/Ag pair may be described by routine methods. For example, the overall location of an epitope may be determined by assessing the ability of the human-like V_{H}H to bind to different fragments or variants of the antigen. The specific amino acids within the antigen that make contact with an epitope may also be determined using routine methods. For example, the human-like V_{H}H and Ag molecules may be combined and the human-like V_{H}H/Ag complex may be crystallized. The crystal structure of the complex may be determined and used to identify specific sites of interaction between the human-like V_{H}H and Ag.

The term “expression” as used herein is defined as the transcription and/or translation of a particular nucleotide sequence.

The term “Fc domain”, or “Fe” as used herein is the crystallizable fragment domain or region obtained from an antibody that comprises the C_{H}2 and C_{H}3 domains of an antibody. In an antibody, the two Fc domains are held together by two or more disulfide bonds and by hydrophobic interactions of the C_{H}3 domains. The Fc domain may be obtained by digesting an antibody with the protease papain.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

The term “germline” or “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of

Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

The term “immunoglobulin single-chain variable domains” (abbreviated herein as “ISVD”, and interchangeably used with “single variable domain”, defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In the latter case, the complementarity determining region (CDR) areas of both V_{H} and V_{L} will contribute to the antigen binding site, i.e. a total of six CDRs will be involved in antigen binding site formation. In view of the above definition, the antigen-binding domain of a conventional four-chain antibody (such as an IgG, IgM, IgA, IgD or IgE molecule; known in the art) or of a Fab fragment, a F(ab)₂ fragment, an Fv fragment such as a disulphide linked Fv or a scFv fragment, or a diabody (all known in the art) derived from such conventional four-chain antibody, would normally not be regarded as an ISVD, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, ISVDs are capable of specifically binding to an epitope of the antigen without pairing with an additional immunoglobulin variable domain. The binding site of an ISVD is formed by a single V_{H}H or V_{H} domain. Hence, the antigen binding site of an ISVD is formed by no more than three CDRs. As such, the single variable domain may be a heavy chain variable domain sequence (e.g., a Vs-sequence or V_{H}H sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An ISVD as used herein is selected from the group consisting of V_{H}Hs, human-like V_{H}Hs, and camelized V_{H}s.

The term “NANOBODY” and “NANOBODIES” as used herein are registered trademarks of Ablynx N.V.

The term “nucleic acid molecule” refers to a polynucleotide.

The term “peptide” typically refers to a polymer composed of less than 41 amino acid residues, related naturally occurring structural variants, and synthetic non-naturally occurring analogs thereof linked via peptide bonds, related naturally occurring structural variants, and synthetic non-naturally occurring analogs thereof.

The term “polynucleotide” as used herein is defined as a chain of nucleotides. Furthermore, nucleic acid molecules are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning and amplification technology, and the like, and by synthetic means. An “oligonucleotide” as used herein refers to a short polynucleotide, typically less than 100 bases in length. RNA and DNA molecules are polynucleotides.

The term “polypeptide” refers to a polymer composed of 41 or more amino acid residues, related naturally occurring structural variants, and synthetic non-naturally occurring analogs thereof linked via peptide bonds, related naturally occurring structural variants, and synthetic non-naturally occurring analogs thereof.

The terms “promoter”, “promoter region”, or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “surface anchor” or “surface anchoring moiety” refers to any polypeptide or peptide that, when fused with an Fc or functional fragment thereof, is expressed and located to the cell surface where a human-like V_{H}H Fc fusion protein can form a pairwise interaction with the Fc or functional fragment thereof attached to the cell surface. An example of a cell surface anchor is a protein such as, but not limited to, SED-1, α-agglutinin, Cwp1, Cwp2, GasI, Yap3, FIoIp1 Crh2, Pir1, Pir4, Tip1, Wpi, Hpwp1, Als3, and Rbt5; for example, Saccharomyces cerevisiae CWP1, CWP2, SED1, or GAS1; Pichia pastoris SP1 or GAS1; or H. polymorpha TIP1. The surface anchor further includes any polypeptide with a signal peptide that when fused to the C-terminus of the Fc or functional fragment thereof (fusion protein) to the endoplasmic reticulum (ER) where it is inserted into the ER membrane via a translocon and is attached to the ER membrane by its hydrophobic C terminus. The hydrophobic C-terminal sequence is then cleaved off and replaced by the GPI-anchor (glycosylphosphatidylinositol). As the fusion protein processes through the secretory pathway, it is transferred via vesicles to the Golgi apparatus and finally to the plasma membrane where it remains attached to a leaflet of the cell membrane.

The term “synthetically generated” with respect to CDR and CDR area sequences refers to CDR sequences which are designed using computer algorithms to identify those amino acids in each CDR or CDR area that may varied over those amino acids that are kept constant to the extent each variable amino acid may be varied. For example, the variable amino acid at a particular position in the CDR or CDR area may be any amino acid except C, or any amino acid except C and M, or any amino acid within a subset of amino acids. A plurality of RNA or DNA molecules encoding V_{H}H are then synthesized wherein each V_{H}H comprises CDRs or CDR areas having a particular combination of variable CDRs and/or CDR areas as determined using the computer algorithms. Thus, a nucleic acid molecule library is constructed in which each nucleic acid molecule independently encodes a particular V_{H}H having a particular combination of CDR and/or CDR area sequences.

The term “target of interest” refers to any molecule, protein, polypeptide, peptide, carbohydrate, nucleic acid, or any other molecule it is desired to have the human-like VHH bind. In general parlance, the target of interest may be referred to as an antigen.

A cell has been “transformed”, “transduced”, or “transfected” by exogenous or heterologous DNA when such DNA has been introduced inside the cell. The introduced RNA or DNA may or may not be integrated (covalently linked) into the genome of the cell. In prokaryotes, yeast, and mammalian cells for example, the introduced DNA may be maintained on an episomal element such as a plasmid. With respect to eukaryotic cells, a stably transformed or transduced cell is one in which the introduced RNA or DNA has become integrated into a chromosome so that it is inherited by daughter cells through chromosome replication. This stability is demonstrated by the ability of the eukaryotic cell to establish cell lines or clones comprised of a population of daughter cells containing the introduced RNA or DNA. A “clone” is a population of cells derived from a single cell or common ancestor by mitosis. A “cell line” is a clone of a primary cell that is capable of stable growth in vitro for many generations.

The term “vector,” as used herein, refers to either a delivery vehicle as described herein or to a vector such as an expression vector.

The term “V_{H}H” as used herein indicates that the heavy chain variable domain is obtained from or originated or derived from a heavy chain antibody. Heavy chain antibodies are functional antibodies that have two heavy chains and no light chains. Heavy chain antibodies exist in and are obtainable from Camelids (e.g., camels and alpacas), members of the biological family Camelidae. V_{H}H antibodies, have originally been described as the antigen binding immunoglobulin (variable) domain of “heavy chain antibodies” (i.e., of “antibodies devoid of light chains”; Hamers-Casterman et al., Nature 363: 446-448 (1993). The term “V_{H}H domain” has been chosen in order to distinguish these variable domains from the heavy chain variable domains that are present in conventional four-chain antibodies (which are referred to herein as “V_{H} domains” or “V_{H}”) and from the light chain variable domains that are present in conventional four-chain antibodies (which are referred to herein as “V_{L} domains” or “V_{L}”). For a further description of V_{H}Hs, reference is made to the review article by Muyldermans (Reviews in Molec. Biotechnol. 74: 277-302, (2001), as well as to the following patent applications, which are mentioned as general background art: WO9404678, WO9504079 and WO9634103 of the Vrije Universiteit Brussel; WO9425591, WO9937681, WO0040968, WO0043507, WO0065057, WO0140310, WO0144301, EP1134231 and WO0248193 of Unilever; WO9749805, WO0121817, WO03035694, WO03054016 and WO03055527 of the Vlaams Instituut voor Biotechnologie (VI B); WO03050531 of Algonomics N.V. and Ablynx N.V.; WO0190190 by the National Research Council of Canada; WO03025020 (=EP 1433793) by the Institute of Antibodies; as well as WO2004041867, WO2004041862, WO2004041865, WO2004041863, WO2004062551, WO2005044858, WO200640153, WO2006079372, WO2006122786, WO 06122787, WO2006122825, WO2008101985, WO2008142164, and WO2015173325 by Ablynx N.V. and the further published patent applications by Ablynx N.V. Reference is also made to the further prior art mentioned in these applications, and in particular to the list of references mentioned on pages 41-43 of the International application WO 06040153, which list and references are incorporated herein by reference.

I. The invention

The present invention provides a synthetic yeast or bacteriophage display platform for in vitro selection of antigen-specific human-like V_{H}H, which may be used for the manufacture of therapeutics for the treatment of diseases or disorders. In this format, human-like V_{H}H genes are synthesized and cloned into a display vector adapted for use in yeast display or bacteriophage display, where they are expressed on the surface of the yeast or bacteriophage, which can then be separated based on antigen binding characteristics.

To optimize the probability of success in identifying high-affinity antibodies, the present invention provides a synthetic yeast or bacteriophage display platform for in vitro selection of antigen-specific human-like V_{H}H. In this format V_{H}H genes are synthesized and cloned into a display vector adapted for use in yeast display or bacteriophage display, where they are expressed on the surface of the yeast or bacteriophage, which can then be separated based on antigen binding characteristics. The human-like V_{H}H libraries used in the present invention confer several advantages over the V_{H}H libraries currently being used: 1) the human-like V_{H}H libraries are based on structural and sequence data to introduce diversity in the CDR1+2 loops only where it may contribute to antigen binding, keeping amino acid sequences close to germline to minimize developability concerns; and 2) the human-like V_{H}H libraries comprise fully human heavy chain variable domain (V_{H}) frameworks 1, 3, and 4 and a human framework 2 substituted with either two or four hallmark alpaca (Camelid) amino acids to eliminate the need to humanize V_{H}H later on as is required using the current V_{H}H libraries in the art,

In particular embodiments, the V_{H}H libraries for use in the yeast display platform may employ a switchable display/secretion system to enable rapid characterization of lead molecules (Shaheen et al., PLoS One 8, e70190 (2013); U.S. Pat. Nos. 9,365,846; 10,106,598).

To demonstrate the utility of the present invention, the inventors conducted human-like V_{H}H discovery campaigns in the switchable display/secretion platform format against three antigens of different sizes and protein classes: a large protein (murine PD1 (mPD-1), a 40 amino acid peptide, and a G-protein coupled receptor (GPCR). As shown in the examples, the inventors were able to isolate many binding human-like V_{H}H for each antigen, targeting distinct epitopes with high affinity (as high as 5 nM). The inventors further tested the mPD-1-specific human-like V_{H}H in a receptor-blocking assay and show that the structure-based libraries yielded mPD-1 binders with functional activity. The present invention V_{H}H libraries are highly productive with the potential to generate high-affinity binders against virtually any target.

The libraries of the present invention may be constructed from any particular Camelid germline V_{H}H amino acid sequence by substituting amino acids beginning in framework 1 on through the end of framework 3 (including germline CDRs) with the amino acids present in the human homologue germline V_{H} amino acid sequence at the corresponding position except for the amino acids at position 44 and 45 (or positions 37, 44, 45, and 47) to produce a human-like V_{H}H germline amino acid sequence. The human-like V_{H}H germline amino acid sequence is then further modified to replace the CDRs with synthetically generated CDRs. The germline CDRs and synthetically generated CDRs may be defined using any of the currently used methods for defining CDR sequences, e.g., including but limited to Kabat, IMGT, AbM, and Chothia numbering schemes. In certain embodiments, only amino acids within the CDR are substituted in other embodiments amino acid substitution may include an amino acid outside the CDR loop, i.e., that is the CDR area. The amino acid substitutions, both location and type, may be determined using a computer algorithm or program. Examples of substituted CDR regions for CDR1, CDR2, and CDR3 are shown in Table 2. Nucleic acid molecules are then synthesized to include each of the substitutions generated by the computer algorithm or program to produce a plurality nucleic acid molecules, each molecule encoding one particular human-like V_{H}H.

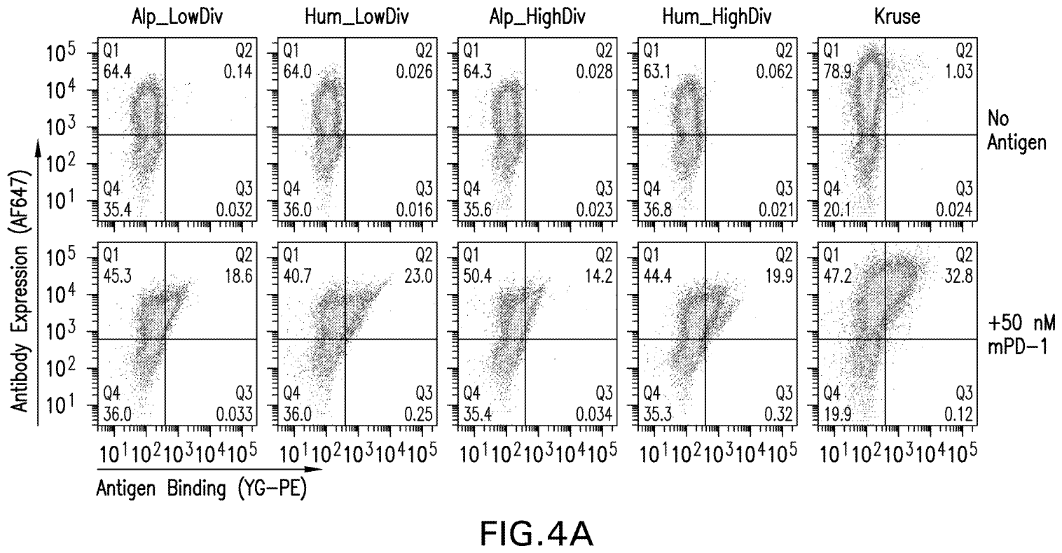

As exemplified in Example 1, a library was designed in which the alpaca IGHV3S53 germline V_{H}H amino acid sequence was aligned with the human IGHV3-23*04 germline V_{H} amino acid sequence from the N-terminus to the end of framework 3 as shown in FIG. 3. The amino acids in the alpaca V_{H}H germline sequence which differed from the amino acids at the corresponding positions in the human IGHV3-23*04 germline V_{H} amino acid sequence with the exception of the amino acids at position 44 and 45 (or positions 37, 44, 45, and 47) to produce a human-like V_{H}H germline amino acid sequence. As shown in the examples, it was discovered that maintaining at least the alpaca amino acids at position 44 and 45 was sufficient to maintain stability of the human-like V_{H}H. The germline CDRs and synthetically generated CDRs for the high diversity library were defined using the IGMT numbering scheme (see FIG. 3 and Table 2) but any numbering scheme may be used. For example, the low diversity library was constructed using the Kabat numbering scheme

Low and high diversity libraries may be constructed, which comprise the particular amino acid substitutions within the three CDR regions as shown in Table 2. The amino acid substitutions, both location and type, were determined using a computer algorithm or program. Nucleic acid molecules are then synthesized to include each of the substitutions generated by the computer algorithm or program to produce a plurality nucleic acid molecules, each molecule encoding one particular human-like V_{H}H.

TABLE 2

| Hum Low | | | |
|---|---|---|---|
| Diversity | CDR1 | CDR2 | CDR3 |
| Kabat | XYXMS | AIXSGGXTYYADSV | XXXXXXXXXXXXXXXFDX |
| | (SEQ ID NO: 9) | KG (SEQ ID NO: 10) | (SEQ ID NO: 11) |
| IMGT | GFTFSXYX | IXSGGXT | ARXXXXXXXXXXXXXXXFDX |
| | (SEQ ID NO: | (SEQ ID NO: 13) | (SEQ ID NO: 14) |
| | 12) | | |
| AbM | GFTFSXYXMS | AIXSGGXTY (SEQ ID | XXXXXXXXXXXXXXXFDX |
| | (SEQ ID NO: | NO: 16) | (SEQ ID NO: 17) |
| | 15) | | |
| Chothia | GFTFSXYX | XSGGX | XXXXXXXXXXXXXXXFDX |
| | (SEQ ID NO: | (SEQ ID NO: 19) | (SEQ ID NO: 20) |
| | 18) | | |
| Hum High | | | |
| Diversity | CDR1 | CDR2 | CDR3 |
| Kabat | XXYXMS (SEQ | XISXXGXXTYYADS | XXXXXXXXXXXXXXXFDX |
| | ID NO: 21) | VKG (SEQ ID NO: 22) | (SEQ ID NO: 23) |
| IMGT | GFTFXXYAM | XISXXGXXT (SEQ ID | ARXXXXXXXXXXXXXXXFDX |
| | X (SEQ ID NO: | NO: 25) | (SEQ ID NO: 26) |
| | 24) | | |
| AbM | GFTFXXYAMX | XISXXGXXTY (SEQ | XXXXXXXXXXXXXXXFDX |
| | (SEQ ID NO: | ID NO: 28) | (SEQ ID NO: 29) |
| | 27) | | |
| Chothia | GFTFXXYAM | XISXXGXX (SEQ ID | XXXXXXXXXXXXXXXFDX |
| | X (SEQ ID NO: | NO: 31) | (SEQ ID NO: 32) |
| | 30) | | |

(1) Amino acids underlined and in bold for SEQ ID Nos: 18. 21, 24, 25, 30, and 31 are in the CDR region but outside the CDR as it is defined for the particular numbering scheme. Thus, the sequences are referred to as corresponding to the CDR area or region, which includes where indicated one amino acid adjacent to the N-terminal end of the CDR and where indicated one, two, or three amino acids adjacent to the C-terminal end of the CDR.
(2) Each X is independently any amino acid except for C.
(3) For Kabat, AbM, and Chothia defined CDR3, X4-X15 may be present or absent.
(4) For IGMT defined CDR3, X6-X17 may be present or absent; X6 refers to the position of X within the whole sequence including AR, not the position of X solely within the series of Xs.

### II. Embodiments of the Invention

The present invention provides a nucleic acid molecule library comprising a plurality of nucleic acid molecules, each nucleic acid molecule encoding a human-like heavy V_{H}H comprising three synthetically generated complementarity determining region (CDR) areas in a human antibody heavy chain variable domain (V_{H}) framework in which the amino acids at each of positions 44 and 45 of the human V_{H} framework are substituted with the amino acids at the corresponding positions of a Camelid heavy chain antibody variable domain (V_{H}H) framework, wherein the amino acid positions are according to Kabat numbering.

In a further embodiment of the nucleic acid molecule library, the human V_{H} framework further includes substitution of each of the amino acids at positions 37 and 47 with the amino acids at corresponding positions 37 and 47 of the Camelid V_{H}H framework, wherein the amino acid positions are according to Kabat numbering.

In a further embodiment of the nucleic acid molecule library, the human V_{H} framework comprises the amino acid sequence of the human V_{H} framework encoded by the human IGHV3-23*04 gene in which the amino acids at positions 44 and 45 of the human V_{H} framework are each substituted with the corresponding amino acids at positions 44 and 45 of the Camelid V_{H}H framework encoded by the alpaca IGHV3S53 gene, wherein the amino acid positions are according to Kabat numbering.

In a further embodiment of the nucleic acid molecule library, the human V_{H} framework comprises the amino acid sequence of the human V_{H} framework encoded by the human IGHV3-23*04 gene and the amino acids at positions 37, 44, 45, and 47 of the human V_{H} framework are each substituted with the corresponding amino acid at positions 37, 44, 45, and 47 of the Camelid V_{H}H framework encoded by the alpaca IGHV3S53 gene, wherein the amino acid positions are according to Kabat numbering.

In a further embodiment of the nucleic acid molecule library, the human-like V_{H}H comprises the amino acid sequence wherein the human-like V_{H}H comprises the amino acid sequence set forth in SEQ ID NO:1, SEQ ID NO: 2, SEQ ID NO: 3, or SEQ ID NO:4.

In a further embodiment of the nucleic acid molecule library, each human-like V_{H}H is a fusion protein wherein the human-like V_{H}H is fused at the C-terminus to a polypeptide or peptide that enables the human-like V_{H}H to be displayed on the outer surface of a host cell or a bacteriophage.

In a further embodiment of the nucleic acid molecule library, the polypeptide is a fragment crystallizable (Fc) region of an immunoglobulin or the coat protein of a bacteriophage and the peptide is a first peptide capable of binding to a second peptide fused to a bacteriophage coat protein that is displayed on the outer surface of the bacteriophage encoded by a second nucleic acid molecule and which is encoded by a second nucleic acid molecule.

The present invention further provides a library of human-like heavy V_{H}H, each V_{H}H comprising three synthetically generated complementarity determining region (CDR) areas in a human antibody heavy chain variable domain (V_{H}) framework in which the amino acids at each of positions 44 and 45 of the human V_{H} framework are substituted with the amino acids at the corresponding positions of a Camelid heavy chain antibody variable domain (V_{H}H) framework, wherein the amino acid positions are according to Kabat numbering.

In a further embodiment of the library, the human V_{H} framework further includes substitution of each of the amino acids at positions 37 and 47 with the amino acid at corresponding positions 37 and 47 of the Camelid V_{H}H framework, wherein the amino acid positions are according to Kabat numbering.

In a further embodiment of the library, the human V_{H} framework comprises the amino acid sequence of the human V_{H} framework encoded by the IGHV3-23*04 gene in which the amino acids at positions 44 and 45 of the human V_{H} framework are each substituted with the corresponding amino acid at positions 44 and 45 of the Camelid V_{H}H framework encoded by the alpaca IGHV3S53 gene, wherein the amino acid positions are according to Kabat numbering.

In a further embodiment of the library, the human V_{H} framework comprises the amino acid sequence of the human V_{H} framework encoded by the IGHV3-23*04 gene and the amino acids at positions 37, 44, 45, and 47 of the human V_{H} framework are each substituted with the corresponding amino acid at positions 37, 44, 45, and 47 of the alpaca V_{H}H framework encoded by the IGHV3S53 gene, wherein the amino acid positions are according to Kabat numbering.

In a further embodiment of the library, wherein the human-like V_{H}H comprises the amino acid sequence wherein the human-like V_{H}H comprises the amino acid sequence set forth in SEQ ID NO:1, SEQ ID NO: 2, SEQ ID NO: 3, or SEQ ID NO:4.

In a further embodiment of the library, the human-like V_{H}H is fused at the C-terminus to a polypeptide or peptide that enables the human-like V_{H}H to be displayed on the outer surface of a host cell or a bacteriophage.

In a further embodiment of the library, the polypeptide is a fragment crystallizable (Fc) region of an immunoglobulin or the coat protein of a bacteriophage and the peptide is a first peptide capable of binding to a second peptide fused to a bacteriophage coat protein that is displayed on the surface of the bacteriophage encoded by a second nucleic acid molecule and which is encoded by a second nucleic acid molecule.

The present invention further provides a human-like heavy V_{H}H comprising three synthetically generated complementarity determining region (CDR) areas in a human antibody heavy chain variable domain (V_{H}) framework in which the amino acids at each of positions 44 and 45 of the human V_{H} framework are substituted with the amino acids at the corresponding positions of a Camelid heavy chain antibody variable domain (V_{H}H) framework, wherein the amino acid positions are according to Kabat numbering.

In a further embodiment of the human-like V_{H}H, the human V_{H} framework further includes substitution of each of the amino acids at positions 37 and 47 with the amino acid at corresponding positions 37 and 47 of the Camelid V_{H}H framework, wherein the amino acid positions are according to Kabat numbering.

In a further embodiment of the human-like V_{H}H, the human V_{H} framework further includes substitution of each of the amino acids at positions 37 and 47 with the amino acid at corresponding positions 37, 44, 45, and 47 of the Camelid V_{H}H framework, wherein the amino acid positions are according to Kabat numbering.

In a further embodiment of the human-like V_{H}H, the human V_{H} framework comprises the amino acid sequence of the human V_{H} framework encoded by the IGHV3-23*04 gene in which the amino acids at positions 44 and 45 of the human V_{H} framework are each substituted with the corresponding amino acid at positions 44 and 45 of the Camelid V_{H}H framework encoded by the alpaca IGHV3S53 gene, wherein the amino acid positions are according to Kabat numbering. In the above embodiments, the substitutions at positions 37, 44, 45, and/or 47 of the V_{H} and V_{H}H framework are located within framework 2. For example, V_{H}H framework 2 of the low diversity alpaca V_{H}H IGHV3S53 V_{H}H represented by the amino acids sequence shown in SEQ ID NO: 5 or the high diversity alpaca V_{H}H IGHV3S53 V_{H}H may be represented by the amino acid sequence shown in SEQ ID NO: 6.

In a further embodiment of the human-like V_{H}H, the human V_{H} framework comprises the amino acid sequence of the human V_{H} framework encoded by the IGHV3-23*04 gene and the amino acids at positions 37, 44, 45, and 47 of the human V_{H} framework are each substituted with the corresponding amino acid at positions 37, 44, 45, and 47 of the alpaca V_{H}H framework encoded by the IGHV3S53 gene, wherein the amino acid positions are according to Kabat numbering.

The present invention further provides a human-like heavy V_{H}H comprising three synthetically generated complementarity determining region (CDR) areas in a human antibody heavy chain variable domain (V_{H}) framework in which the amino acids at each of positions 44 and 45 of the human V_{H} framework are Gln and Arg, respectively.

In a further embodiment of the human-like V_{H}H, the human V_{H} framework further includes substitution of each of the amino acids at positions 37 and 47 are Tyr and Leu, respectively.

In a further embodiment of the human-like V_{H}H, the human V_{H} framework further includes substitution of each of the amino acids at positions 37, 44, 45, and 47 of the human V_{H} framework are Tyr, Gln, Arg, and Leu, respectively.

In a further embodiment of the human-like V_{H}H, the human V_{H} framework comprises the amino acid sequence of the human V_{H} framework encoded by the IGHV3-23*04 gene in which the amino acids at positions 44 and 45 of the human V_{H} framework are Gln and Arg, respectively.

In a further embodiment of the human-like V_{H}H, the human V_{H} framework comprises the amino acid sequence of the human V_{H} framework encoded by the IGHV3-23*04 gene and the amino acids at positions 37, 44, 45, and 47 of the human V_{H} framework are Tyr, Gln, Arg, and Leu, respectively. The human V_{H} framework and Camelid V_{H}H framework each comprises four frameworks and three CDRs in the following sequence: (framework 1)-(CDR1)-(framework 2)-(CDR2)-(framework 3)-(CDR3)-(framework 4).

Thus, in each of the foregoing inventions and embodiments, the amino acid at position 37, 44, 45, and/or 47 of the human V_{H} framework following substitution with the amino acid at the corresponding position in the Camelid V_{H}H, when present, is Tyr, Gln, Arg, and/or Leu, respectively.

In particular embodiments, the human V_{H} framework comprises Gln and Arg at positions 44 and 45, respectively, wherein the amino acids at the remainder of the positions in the amino acid sequence of the human V_{H} framework are native to the human V_{H} framework, for example, the human V_{H} framework encoded by the IGHV3-23*04 gene.

In particular embodiments, the human V_{H} framework comprises Tyr, Gln, Arg, and Leu at positions 37, 44, 45, and 47, respectively, wherein the amino acids at the remainder of the positions in the human V_{H} framework are native to the human V_{H} framework, for example, the human V_{H} framework encoded by the IGHV3-23*04 gene. In particular embodiments, the amino acids in the remainder of human V_{H} framework 2 correspond to the amino acids present in the human V_{H} framework 2. In further embodiments, human V_{H} frameworks 1, 3, and 4 comprise the amino acid sequences native to the human V_{H} framework 1, 3, and 4 of the human V_{H} framework. In particular embodiments, human V_{H} frameworks 1, 3, and 4 may comprise 1, 2, 3, 4, or 5 amino acid substitutions.

In particular embodiments, the amino acids at position 37, 44, 45, and/or 47 of the human V_{H} framework 2 following substitution with the amino acid at the corresponding position in the Camelid V_{H}H, when present, are Tyr, Gln, Arg, and/or Leu, respectively. In particular embodiments, the amino acids in the remainder of framework 2 correspond to the amino acids present in the human V_{H} framework 2. In further embodiments, human V_{H} frameworks 1, 3, and 4 comprise the amino acid sequences native to the human V_{H} framework 1, 3, and 4 of the human V_{H} framework. In particular embodiments, human V_{H} frameworks 1, 3, and 4 may comprise 1, 2, 3,4, or 5 amino acid substitutions.

In particular embodiments, the human V_{H} framework 2 comprises Gln and Arg at positions 44 and 45, respectively, wherein the amino acids at the remainder of the positions in the amino acid sequence of human V_{H} framework 2 are native to the human V_{H} framework, for example, the human V_{H} framework 2 encoded by the IGHV3-23*04 gene.

In further embodiments, the human V_{H} framework 2 comprises Tyr, Gln, Arg, and Leu at positions 37, 44, 45, and 37, respectively, wherein the amino acids at the remainder of the positions in the amino acid sequence of human V_{H} framework 2 are native to the human V_{H} framework 2, for example, the human V_{H} framework 2 encoded by the IGHV3-23*04 gene of which comprises the amino acid sequence.

In further embodiments, the human V_{H} framework 2 comprises Gln and Arg at positions 44 and 45, respectively, wherein the amino acids at the remainder of the positions in the amino acid sequence of human V_{H} framework 2 and the amino acid sequences of frameworks 1 and 3 are native to the human V_{H} framework, for example, the human V_{H} frameworks encoded by the IGHV3-23*04 gene.

In further embodiments, the human V_{H} framework 2 comprises Tyr, Gln, Arg, and Leu at positions 37, 44, 45, and 37, respectively, wherein the amino acids at the remainder of the positions in the amino acid sequence of human V_{H} framework 2 and frameworks 1 and 3 are native to the human V_{H} framework, for example, the human V_{H} frameworks encoded by the IGHV3-23*04 gene of which comprises the amino acid sequence.

In further embodiments, the human V_{H} framework 2 comprises Gln and Arg at positions 44 and 45, respectively, wherein the amino acids at the remainder of the positions in the amino acid sequence of human V_{H} framework 2 and the amino acid sequences of frameworks 1, 3, and 4 are native to the human V_{H} framework, for example, the human V_{H} frameworks encoded by the IGHV3-23*04 gene.

In further embodiments, the human V_{H} framework 2 comprises Tyr, Gln, Arg, and Leu at positions 37, 44, 45, and 37, respectively, wherein the amino acids at the remainder of the positions in the amino acid sequence of human V_{H} framework 2 and frameworks 1, 3, and 4 are native to the human V_{H} framework, for example, the human V_{H} frameworks encoded by the IGHV3-23*04 gene.

While the boundary between the CDRs and the frameworks will vary depending on the method used for defining the CDRs, e.g., Kabat, IMGT, AbM, Chothia, and the like, positions 37, 44, 45, and 47 reside within framework 2 regardless of the method used to define the CDRs.

In a further embodiment of the human-like V_{H}H, wherein the human-like V_{H}H comprises the amino acid sequence wherein the human-like V_{H}H comprises the amino acid sequence set forth in SEQ ID NO:1, SEQ ID NO: 2, SEQ ID NO: 3, or SEQ ID NO:4.

In a further embodiment of the human-like V_{H}H, the human-like V_{H}H is fused at the C-terminus to a polypeptide or peptide that enables the human-like V_{H}H to be displayed on the outer surface of a host cell or a bacteriophage.

In a further embodiment of the human-like V_{H}H, the polypeptide is a fragment crystallizable (Fc) region of an immunoglobulin or the coat protein of a bacteriophage and the peptide is a first peptide capable of binding to a second peptide fused to a bacteriophage coat protein that is displayed on the surface of the bacteriophage encoded by a second nucleic acid molecule and which is encoded by a second nucleic acid molecule.

The present invention further provides a vector comprising a nucleic acid molecule encoding the human-like V_{H}H of any one of the foregoing embodiments. The present invention further provides a host cell comprising the vector.

In a further embodiments of the host cell, the host cell further includes a vector that encodes an Fc region of an immunoglobulin fused to a cell surface anchoring moiety that enables the Fc fusion protein to be displayed on the outer surface of the host cell.

In a further embodiments of the host cell, the host cell is a yeast or filamentous fungus. In a further embodiments of the host cell, the host cell is a Saccharomyces cerevisiae or Pichia pastoris strain.

The present invention further provides a library of host cells comprising the library of nucleic acid molecules that encode the human-like V_{H}H disclosed herein.

The present invention further provides a bacteriophage comprising a nucleic acid molecule encoding the human-like V_{H}H of any one embodiments of the nucleic acid molecules fused to a bacteriophage coat protein or to a first peptide that is capable of binding to a second peptide fused to a bacteriophage coat protein that is displayed on the outer surface of the bacteriophage and which is encoded by a second nucleic acid molecule. The present invention further provides a library of bacteriophage comprising the library of nucleic acid molecules that encode the human-like V_{H}H disclosed herein.

The present invention further provides a display system for displaying a human-like heavy V_{H}H on the outer surface of a host cell comprising

(a) a plurality of first expression vectors, each first expression vector comprising a nucleic acid molecule encoding (i) a human-like V_{H}H fusion protein comprising three synthetically generated complementarity determining region (CDR) areas in a human antibody heavy chain variable domain (V_{H}) framework in which the amino acids at each of positions 44 and 45 of the human V_{H} framework are substituted with the amino acids at the corresponding positions of a Camelid heavy chain antibody variable domain (V_{H}H) framework, wherein the amino acid positions are according to Kabat numbering, and (ii) a first Fc polypeptide;

(b) a multiplicity of second expression vectors, each second expression vector comprising a nucleic acid molecule encoding a bait polypeptide comprising a second Fc polypeptide fused to a polypeptide or peptide that enables the second Fc polypeptide to be displayed on the outer surface of a host cell, the first and second Fc polypeptides acting, when the human-like V_{H}H fusion protein is produced in the host cell, to cause the display of the human-like V_{H}H fusion protein via pairwise interaction between the first and second Fc polypeptides; and

(c) host cells for transforming with the plurality of first expression vectors and multiplicity of second expression vectors.

In a further embodiment of the display system, the human V_{H} framework further includes substitution of each of the amino acids at positions 37 and 47 with the amino acids at corresponding positions 37 and 47 of the Camelid V_{H}H framework, wherein the amino acid positions are according to Kabat numbering.

In a further embodiment of the display system, the human V_{H} framework comprises the amino acid sequence of the human V_{H} framework encoded by the human IGHV3-23*04 gene in which the amino acids at positions 44 and 45 of the human V_{H} framework are each substituted with the corresponding amino acid at positions 44 and 45 of the Camelid V_{H}H framework encoded by the alpaca IGHV3S53 gene, wherein the amino acid positions are according to Kabat numbering.

In a further embodiment of the display system, the human V_{H} framework comprises the amino acid sequence of the human V_{H} framework encoded by the IGHV3-23*04 gene and the amino acids at positions 37, 44, 45, and 47 of the human V_{H} framework are each substituted with the corresponding amino acid at positions 37, 44, 45, and 47 of the Camelid V_{H}H framework encoded by the alpaca IGHV3S53 gene, wherein the amino acid positions are according to Kabat numbering.

In a further embodiment of the display system, each human-like V_{H}H comprises the amino acid sequence wherein the human-like V_{H}H comprises the amino acid sequence set forth in SEQ ID NO:1, SEQ ID NO: 2, SEQ ID NO: 3, or SEQ ID NO:4.

In a further embodiment of the display system, the host cell is a yeast or filamentous fungus.

In a further embodiment of the display system, the host cell is a Saccharomyces cerevisiae or Pichia pastoris strain.

The present invention further provides a bacteriophage display system for displaying a human-like heavy V_{H}H on the outer surface of a bacteriophage, comprising a plurality of bacteriophage, each bacteriophage comprising a nucleic acid molecule encoding a fusion protein comprising

(a) comprising three synthetically generated complementarity determining region (CDR) areas in a human antibody heavy chain variable domain (V_{H}) framework in which the amino acids at each of positions 44 and 45 of the human V_{H} framework are substituted with the amino acids at the corresponding positions of a Camelid heavy chain antibody variable domain (V_{H}H) framework, wherein the amino acid positions are according to Kabat numbering, and

(b) a bacteriophage coat protein or a first peptide that is capable of binding to a second peptide fused to a bacteriophage coat protein that is displayed on the outer surface of the bacteriophage and which is encoded by a second nucleic acid molecule provided by a helper bacteriophage.

In a further embodiment of the bacteriophage display system, the human V_{H} framework further includes substitution of each of the amino acids at positions 37 and 47 with the amino acid at corresponding positions 37 and 47 of the Camelid V_{H}H framework, wherein the amino acid positions are according to Kabat numbering.

In a further embodiment of the bacteriophage display system, the human V_{H} framework comprises the amino acid sequence of the human V_{H} framework encoded by the IGHV3-23*04 gene in which the amino acids at positions 44 and 45 of the human V_{H} framework are each substituted with the corresponding amino acid at positions 44 and 45 of the Camelid V_{H}H framework encoded by the alpaca IGHV3S53 gene, wherein the amino acid positions are according to Kabat numbering.

In a further embodiment of the bacteriophage display system, the human V_{H} framework comprises the amino acid sequence of the human V_{H} framework encoded by the IGHV3-23*04 gene and the amino acids at positions 37, 44, 45, and 47 of the human V_{H} framework are each substituted with the corresponding amino acid at positions 37, 44, 45, and 47 of the Camelid V_{H}H framework encoded by the alpaca IGHV3S53 gene, wherein the amino acid positions are according to Kabat numbering.

In a further embodiment of the bacteriophage display system, wherein the human-like V_{H}H comprises the amino acid sequence wherein the human-like V_{H}H comprises the amino acid sequence set forth in SEQ ID NO:1, SEQ ID NO: 2, SEQ ID NO: 3, or SEQ ID NO:4.

The present invention further provides a method for identifying a human-like heavy V_{H}H that binds a target of interest, the method comprising

(a) providing a plurality of transformed host cells comprising
(i) a plurality of first expression vectors, each first expression vector comprising a nucleic acid molecule encoding a human-like V_{H}H fusion protein comprising
   (aa) comprising three synthetically generated complementarity determining region (CDR) areas in a human antibody heavy chain variable domain (V_{H}) framework in which the amino acids at each of positions 44 and 45 of the human V_{H} framework are substituted with the amino acids at the corresponding positions of a Camelid heavy chain antibody variable domain (V_{H}H) framework, wherein the amino acid positions are according to Kabat numbering, and
   (bb) a first Fc polypeptide; and
(ii) a multiplicity of second expression vectors, each second expression vector comprising a nucleic acid molecule encoding a bait polypeptide comprising a second Fc polypeptide fused to a polypeptide or peptide that enables the second Fc polypeptide to be displayed on the outer surface of a host cell, the first and second Fc polypeptides acting, when the human-like V_{H}H fusion protein is produced in the host cell, to cause the display of the human-like V_{H}H fusion protein via pairwise interaction between the first and second Fc polypeptides;

(b) cultivating the transformed host cells under conditions to induce expression of the human-like V_{H}H fusion proteins and the bait polypeptide to produce induced host cells in which the bait polypeptide is displayed on the outer surface of the transformed host cells and the human-like V_{H}H fusion protein is in a pairwise interaction with the bait polypeptide;

(c) contacting the induced host cells with the target of interest conjugated to a detection moiety; and

(d) detecting the detection moiety and selecting the host cells that express the human-like V_{H}H fusion protein that binds the target of interest.

In a further embodiment of the method, the human V_{H} framework further includes substitution of each of the amino acids at positions 37 and 47 with the amino acid at corresponding positions 37 and 47 of the Camelid V_{H}H framework, wherein the amino acid positions are according to Kabat numbering.

In a further embodiment of the method, the V_{H} framework comprises the amino acid sequence of the human V_{H} framework encoded by the IGHV3-23*04 gene in which the amino acids at positions 44 and 45 of the human V_{H} framework are each substituted with the corresponding amino acid at positions 44 and 45 of the Camelid V_{H}H framework encoded by the alpaca IGHV3S53 gene, wherein the amino acid positions are according to Kabat numbering.

In a further embodiment of the method, the V_{H} framework comprises the amino acid sequence of the human V_{H} framework encoded by the IGHV3-23*04 gene and the amino acids at positions 37, 44, 45, and 47 of the human V_{H} framework are each substituted with the corresponding amino acid at positions 37, 44, 45, and 47 of the Camelid V_{H}H framework encoded by the alpaca IGHV3S53 gene, wherein the amino acid positions are according to Kabat numbering.

In a further embodiment of the method, each human-like V_{H}H comprises the amino acid sequence wherein the human-like V_{H}H comprises the amino acid sequence set forth in SEQ ID NO:1, SEQ ID NO: 2, SEQ ID NO: 3, or SEQ ID NO:4.

In a further embodiment of the method, the host cell is a yeast or filamentous fungus. In a further embodiment of the method, the host cell is a Saccharomyces cerevisiae or Pichia pastoris strain.

The present invention further provides a method for identifying a human-like heavy V_{H}H that binds a target of interest, the method comprising

(a) providing a recombinant bacteriophage library, each bacteriophage comprising a nucleic acid molecule encoding a fusion protein comprising a bacteriophage coat protein fused to a human-like V_{H}H comprising three synthetically generated complementarity determining region (CDR) areas in a human antibody heavy chain variable domain (V_{H}) framework in which the amino acids at each of positions 44 and 45 of the human V_{H} framework are substituted with the amino acids at the corresponding positions of a Camelid heavy chain antibody variable domain (V_{H}H) framework, wherein the amino acid positions are according to Kabat numbering, and displaying the fusion protein on the outer surface thereof

(b) contacting the recombinant bacteriophage library with the target of interest immobilized on a solid support;

(c) removing the recombinant bacteriophage in the library that do not bind the target of interest and eluting the recombinant bacteriophage bound to the target of interest to provide recombinant bacteriophage that bind the target of interest;

(d) repeating steps (b) and (c) one to three times to provide a population of recombinant bacteriophage enriched for recombinant bacteriophage that bind the target of interest; and

(d) determining the amino acid sequence of the human-like V_{H}H to provide the human-like V_{H}H that binds the target of interest.

In a further embodiment of the method, the human V_{H} framework further includes substitution of each of the amino acids at positions 37 and 47 with the amino acid at corresponding positions 37 and 47 of the Camelid V_{H}H framework, wherein the amino acid positions are according to Kabat numbering.

In a further embodiment of the method, the human V_{H} framework comprises the amino acid sequence of the human V_{H} framework encoded by the IGHV3-23*04 gene in which the amino acids at positions 44 and 45 of the human V_{H} framework are each substituted with the corresponding amino acid at positions 44 and 45 of the Camelid V_{H}H framework encoded by the alpaca IGHV3S53 gene, wherein the amino acid positions are according to Kabat numbering.

In a further embodiment of the method, the human V_{H} framework comprises the amino acid sequence of the human V_{H} framework encoded by the IGHV3-23*04 gene and the amino acids at positions 37, 44, 45, and 47 of the human V_{H} framework are each substituted with the corresponding amino acid at positions 37, 44, 45, and 47 of the Camelid V_{H}H framework encoded by the alpaca IGHV3S53 gene, wherein the amino acid positions are according to Kabat numbering.

In a further embodiment of the method, the human-like V_{H}H comprises the amino acid sequence wherein the human-like V_{H}H comprises the amino acid sequence set forth in SEQ ID NO:1, SEQ ID NO: 2, SEQ ID NO: 3, or SEQ ID NO:4.

### Yeast, Filamentous Fungi, and Bacterial Surface Display

More recently, target-specific V_{H}H have also been selected by bacterial (Wendel et al., Microb. Cell fact. 15:71 (2016)) or yeast (Kruse et al., Nature 504:101-106 (2013); Rychaert et al., J. Biotechnol. 15: 93-98 (2010); McMahon et al., Nat. Struct. Mol. Biol. 25:289-296 (2018) surface display followed by cell sorting. The major advantage of cell-surface display is the compatibility of these methods with the quantitative and multi-parameter analysis offered by flow cytometry. In this connection, each individual cell of the library can be investigated one by one for the display level of the cloned affinity reagent and its antigen occupancy in real time, Nat. Biotechnol. 15:553-557 (1997)), under well-controlled conditions including buffer composition, pH, temperature and antigen concentration. Accordingly, high-throughput fluorescence-activated cell sorting (FACS) allows the selection and recovery of separate cell populations, displaying binders with different predesignated properties.

Saccharomyces cerevisiae cells, displaying up to hundred thousand copies of a unique affinity reagent fused to the N-terminal end of the Aga2p subunit (Boder & Wittrup, Ibid.) are now widely used as an alternative for display methods based on filamentous phage. Uchański et al. in Sci. reps. 9:382 (2019) disclose a yeast display system wherein each V_{H}H is fused at its C-terminus to the N-terminus of Aga2p. The display level of a cloned V_{H}H on the surface of an individual yeast cell can be monitored through a covalent fluorophore that is attached in a single enzymatic step to an orthogonal acyl carrier protein (ACP) tag³⁵.

The switchable display/secretion system is another yeast display system, which is disclosed in Shaheen et al., PLoS One 8, e70190 (2013); U.S. Pat. Nos. 9,365,846; and, 10,106,598. Previous methods relied on capturing antibodies on the cell surface following secretion in culture medium. The switchable display/secretion system avoids cross-contamination between clones within the same culture by capturing the antibody prior to secretion. Advantageously, embodiments of the present invention allow co-secretion of the displayed molecule allowing further in vitro analysis. Thus, the switchable display/secretion system enables rapid characterization of lead molecules.

The switchable display/secretion system comprises a yeast or filamentous host cell comprising a nucleic acid molecule encoding bait comprising an Fc immunoglobulin domain or functional fragment thereof sufficient to for an Fc pairwise interaction fused at the C-terminus to a surface anchor polypeptide or functional fragment thereof operably linked to a regulatable promoter; and a diverse population of nucleic acid molecules encoding human-like V_{H}Hs fused to an Fc domain or functional fragment thereof, each nucleic acid molecule operably linked to a regulatable promoter (e.g., the nucleic acid molecule library disclosed herein. In particular embodiments, the regulatable promoter is selected from the group consisting of a GUT1 promoter, a GADPH promoter, a GAL promoter, or a PCK1 promoter.

Regulatory sequences which may be used in the practice of the yeast display methods disclosed herein include signal sequences, promoters, and transcription terminator sequences. It is generally preferred that the regulatory sequences used be from a species or genus that is the same as or closely related to that of the host cell or is operational in the host cell type chosen. Examples of signal sequences include those of Saccharomyces cerevisiae invertase; the Aspergillus niger amylase and glucoamylase; human serum albumin; Kluyveromyces maxianus inulinase; and Pichia pastoris mating factor and Kar2. Signal sequences shown herein to be useful in yeast and filamentous fungi include, but are not limited to, the alpha mating factor presequence and preprosequence from Saccharomyces cerevisiae; and signal sequences from numerous other species.

Examples of promoters include promoters from numerous species, including but not limited to alcohol-regulated promoter, tetracycline-regulated promoters, steroid-regulated promoters (e.g., glucocorticoid, estrogen, ecdysone, retinoid, thyroid), metal-regulated promoters, pathogen-regulated promoters, temperature-regulated promoters, and light-regulated promoters. Specific examples of regulatable promoter systems well known in the art include but are not limited to metal-inducible promoter systems (e.g., the yeast copper-metallothionein promoter), plant herbicide safner-activated promoter systems, plant heat-inducible promoter systems, plant and mammalian steroid-inducible promoter systems, Cym repressor-promoter system (Krackeler Scientific, Inc. Albany, N.Y.), RheoSwitch System (New England Biolabs, Beverly Mass.), benzoate-inducible promoter systems (See WO2004/043885), and retroviral-inducible promoter systems. Other specific regulatable promoter systems well-known in the art include the tetracycline-regulatable systems (See for example, Berens & Hillen, Eur J Biochem 270: 3109-3121 (2003)), RU 486-inducible systems, ecdysone-inducible systems, and kanamycin-regulatable system. Yeast-specific promoters include but are not limited to the Saccharomyces cerevisiae TEF-1 promoter, Pichia pastoris GAPDH promoter, Pichia pastoris GUT1 promoter, PMA-1 promoter, Pichia pastoris PCK-1 promoter, and Pichia pastoris AOX-1 and AOX-2 promoters. For temporal expression of the GPI-IgG capture moiety and the immunoglobulins, the Pichia pastoris GUT1 promoter operably linked to the nucleic acid molecule encoding the GPI-IgG capture moiety and the Pichia pastoris GAPDH promoter operably linked to the nucleic acid molecule encoding the immunoglobulin are shown in the examples herein to be useful. In particular embodiments, the regulatable promoter is selected from the group consisting of a GUT1 promoter, a GADPH promoter, a GAL promoter, or a PCK1 promoter.

Examples of transcription terminator sequences include transcription terminators from numerous species and proteins, including but not limited to the Saccharomyces cerevisiae cytochrome C terminator; and Pichia pastoris ALG3 and PMA1 terminators.

Host cells useful for display include Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa. Various yeasts, such as K. lactis, Pichia pastoris, Pichia methanolica, and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale.

Host cells displaying human-like VHH that bind a target of interest can be identified and isolated by incubating the host cells with the target of interest conjugated to a detectable moiety.

The following examples are intended to promote a further understanding of the present invention.

### General Methods

Standard methods in molecular biology are described in Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel, et al. (2001) Current Protocols in Molecular Biology, Vols.1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (e.g., Coligan, et al. (2000) Current Protocols in Protein Science, Vol. 1, John Wiley and Sons, Inc., New York). Chemical analysis, chemical modification, post-translational modification, production of fusion proteins, and glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (e.g., Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4. John Wiley, Inc., New York).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (e.g., Molecular Probes (2003) Catalogue, Molecular Probes, Inc., Eugene, Oreg.; Sigma-Aldrich (2003) Catalogue, St. Louis, Mo.).

### EXAMPLE 1

This example describes the structure- and sequence-based design of synthetic single-domain antibody libraries of the present invention.

### Structure-Based Design

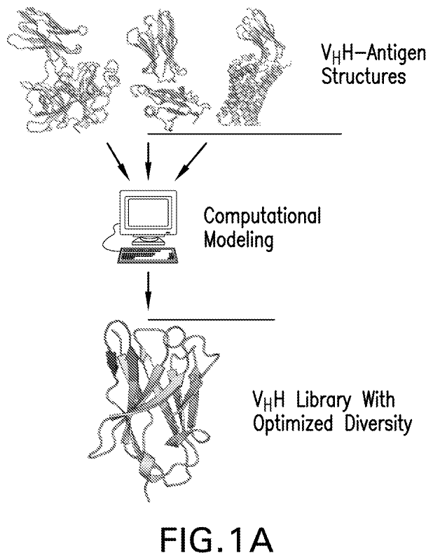

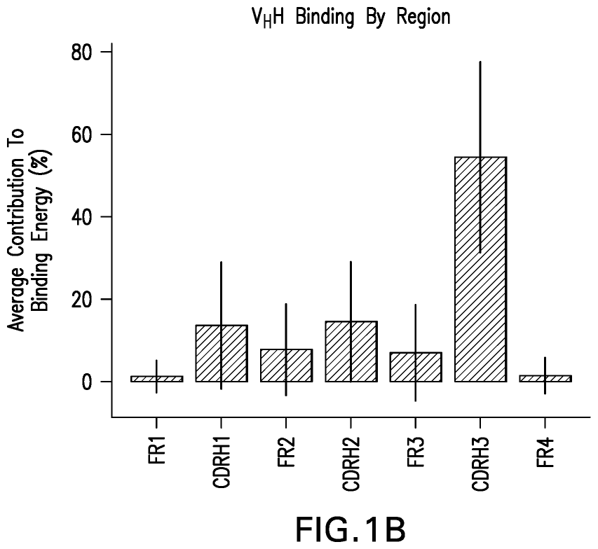

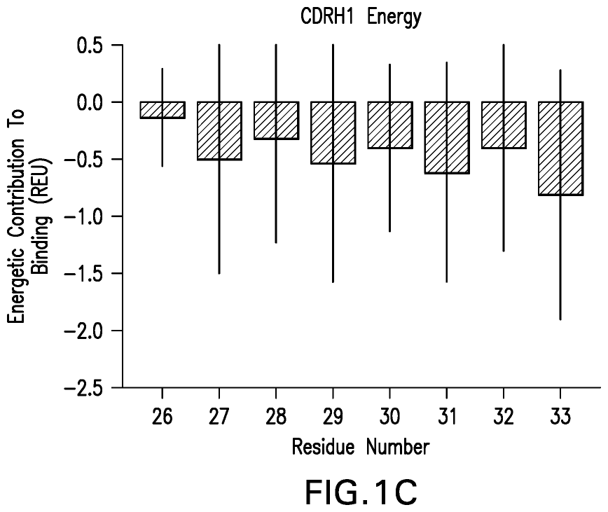

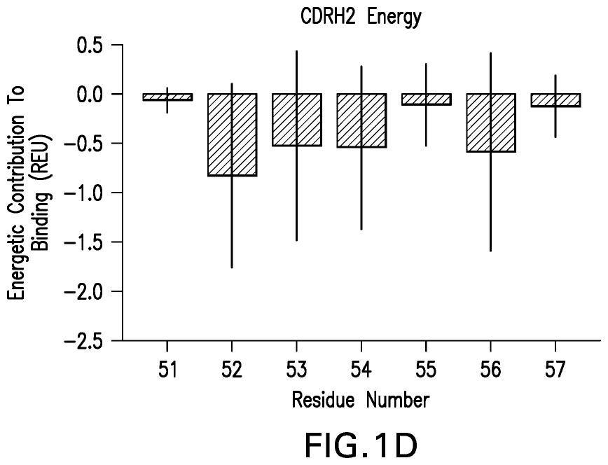

V_{H}H-antigen complexes available in the Protein DataBank were identified and filtered for unique V_{H}H with sub-3.5 Å resolution and protein or peptide antigen. This yielded a total of 208 complexes. The Rosetta protein modeling software¹⁹ was then used to measure the predicted binding energy of each complex and the binding contributions were subdivided by region, to analyze how V_{H}H typically engage their targets (FIG. 1A). This was accomplished by measuring binding energy on a per-residue basis, then dividing the contribution by residues from a given region over binding energy over the entire V_{H}H. We found that, on average, almost 60% of the total binding energy was contributed by the CDRH3 loop, with CDRH1 and CDRH2 contributing roughly equal amounts (˜15% each) to the binding energy (FIG. 1B). Surprisingly, there was a larger contribution from the framework 2 and 3 regions than expected—in fact we observed many individual cases where the binding energy was dominated by framework residues. However, to maintain stability of the molecule, we decided to leave these residues untouched in library design. Therefore, we decided to focus equally on the CDRH1 and CDRH2 loops.

When designing a synthetic library, mutations need to be added strategically to maximize possibility of antigen interaction without destabilization. Therefore, we analyzed which positions along the CDRH1 and CDRH2 tend to contribute most strongly on an energetic basis to antigen interaction, to determine which are the highest priority to diversify (FIG. 1C, FIG. 1D). We observed that the strongest interaction tended to involve residues 31 and 33 (Kabat numbering used throughout) on the CDRH1 and residues 52 and 56 on the CDRH2. We also observed that several positions very rarely contributed to antigen binding, such as residue 26 on the CDRH1 and residues 51, 55, and 57 on the CDRH2. This fits with the understanding of the role of residue 51 in contributing to the hydrophobic core of the V_{H}H²⁰, and the highly conserved nature of residue 26²¹. From this analysis we prioritized residues 31, 33, 52, and 56 as candidates for diversification.

### Sequence-Based Library Design

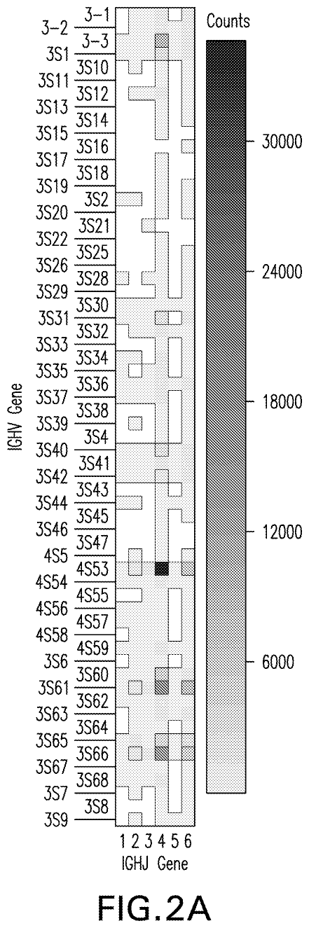

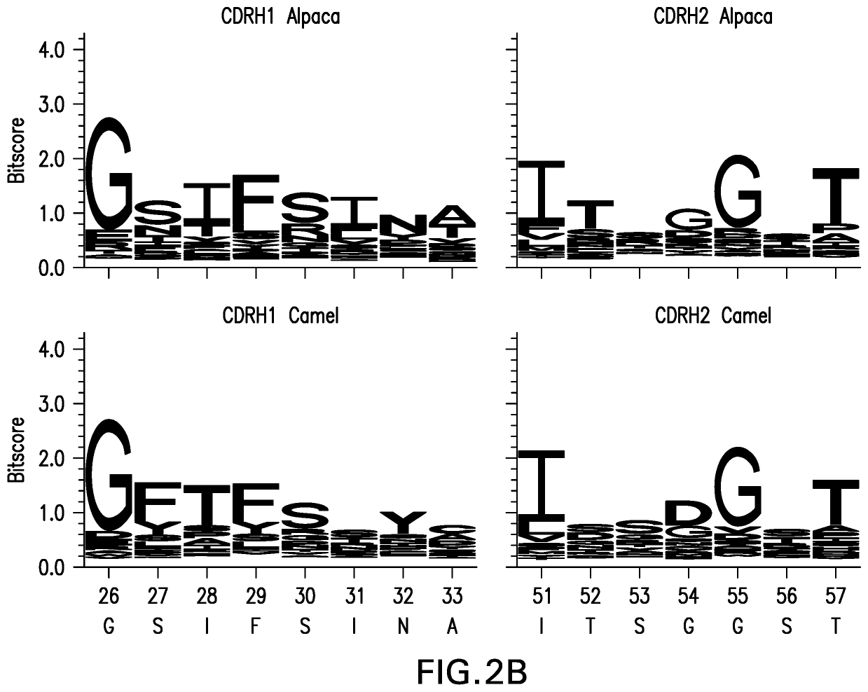

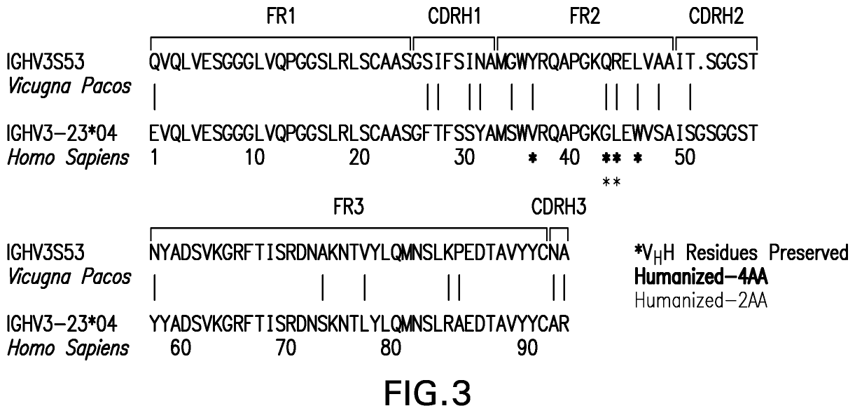

In addition to structural analysis, we sought to analyze properties of V_{H}H repertoires from next-generation sequencing (NGS) datasets. We expected that the amino acid profiles in the CDRH1 and CDRH2 would shed light on which residues are most frequently available for antigen interaction and which are strictly conserved. We identified two publicly available NGS datasets of V_{H}H from alpaca²² and Bactrian camel²³, and downloaded and processed the raw data to analyze V_{H}H properties. We found that the alpaca repertoire was highly restricted in IGHV and IGHJ usage, with over 50% of sequences being encoded by IGHV3S53 and IGHJ4 (FIG. 2A). The data were first de-deduplicated by CDRH3 before germline analysis, to exclude the possibility of a few dominant clones biasing the distribution. Since the IGHV3S53-IGHJ4 germline combination was so dominant, we chose to use this framework as the basis for the synthetic libraries. We next analyzed the CDRH1 and CDRH2 amino acid profiles in sequences encoded by IGHV3S53 and IGHJ4 (n=110,416 for alpaca, n=19,222 for camel). Although the germline gene usage was highly conserved, CDRH1 and CDRH2 amino acid sequences from alpaca and camel were highly variable (FIG. 2B, panels B-E). Alpaca and camel datasets shared similar patterns of conservation, with G26 on the CDRH1 and I51, G55, and T57 on CDRH2 being highly conserved. This agreed with the structural analysis which showed that these residues tended to contribute little to antigen binding (FIG. 1C, FIG. 1D). Overall the sequence and structural data agreed on the importance of maintaining residue identity at positions critical for V_{H}H structure. Based on these two orthogonal analyses, positions 31, 33, 52, and 56 were prioritized for diversification as residues most likely to contribute to antigen recognition.

### Humanization

In addition to the alpaca IGHV3S53 framework used to construct the synthetic libraries, we designed a humanized framework that would eliminate the need for humanization after lead identification. We aligned the alpaca IGHV3S53 gene to the closest human homolog, IGHV3-23*04 (FIG. 3). There were a total of 19 amino acid differences between IGHV3S53 and IGHV3-23*04 (FIG. 3, vertical lines), plus one amino acid insertion in the CDRH2 of IGHV3-23*04. A previous study of V_{H}H humanization showed that two hallmark amino acids in the framework 2 (FR2) are critical for V_{H}H stability (Q44/R45; FIG. 3), with an additional two amino acids contributing to antigen affinity but not required for stability (Y37/L47; FIG. 3)²⁴. We therefore decided to build two humanized frameworks, one maintaining the two hallmark FR2 amino acids and one maintaining four FR2 amino acids. We refer to these two frameworks as Humanized-2AA and Humanized-4AA, respectively.

### Library Construction

Based on the previously described design principles, we designed four V_{H}H libraries for synthesis (Table 3).

TABLE 3
Final library design

| | | | CDR1 + 2 | |
|---|---|---|---|---|
| | | CDR1 + 2 | theoretical | Transformed |
| Library | Framework | diversity | diversity | library size |
| Alp_LowDiv | Alpaca | Low | 6.5 × 10⁵ | 1.2 × 10⁹ |
| HumLowDiv | Humanized-4AA | Low | 6.5 × 10⁵ | 1.5 × 10⁹ |
| AlpHighDiv | Alpaca | High | 1.5 × 10¹² | 0.9 × 10⁹ |
| HumHighDiv | Humanized-2AA | Medium | 1.6 × 10⁷ | 1.1 × 10⁹ |
| Kruse* | Llama consensus | High | 2.3 × 10¹⁰ | 1 × 10⁹ |

Four libraries were synthesized and tested, differing in their level of CDRH1 and CDRH2 diversity, and in the framework
*A library from McMahon, et al., Nat. Struct. Mol. Biol. 25, 289-296 (2018) that was used as a control.

These synthetic libraries differed in using either fully alpaca (Alp) or partially humanized (Hum) frameworks, and in the level of diversity in the CDRH1 and CDRH2 (HighDiv for high diversity or LowDiv for low diversity). In addition to the structurally-guided low diversity libraries described above, we made two high diversity libraries randomizing the full CDRH1 and CDRH2 loops, using either degenerate codons covering a minimalist set of amino acids (Alp_HighDiv) or spiked nucleotide ratios to bias towards germline codons (Hum_HighDiv). A common CDRH3 library consisting of fragments 6-18 amino acids in length (Kabat CDRH3 definition) was spliced to each framework using overlap extension PCR (see Methods in Example 2 for details). The fully assembled V_{H}H gene fragment was then transformed into yeast and cloned into a display vector via homologous recombination. The display vector consisted of V_{H}H fused to human Fc to enable a switchable display/secretion system¹⁸, with an HA peptide tag to enable detection of V_{H}H expression on the yeast surface. The high efficiency transformation protocol was able to achieve library sizes of 10⁹ (Table 3). In addition to the four synthetic libraries designed herein, we included a synthetic library designed by McMahon, et al.¹⁴ derived from llama genes IGHV1S1-IGHV1S1S5 (Kruse library) to compare our synthetic libraries.

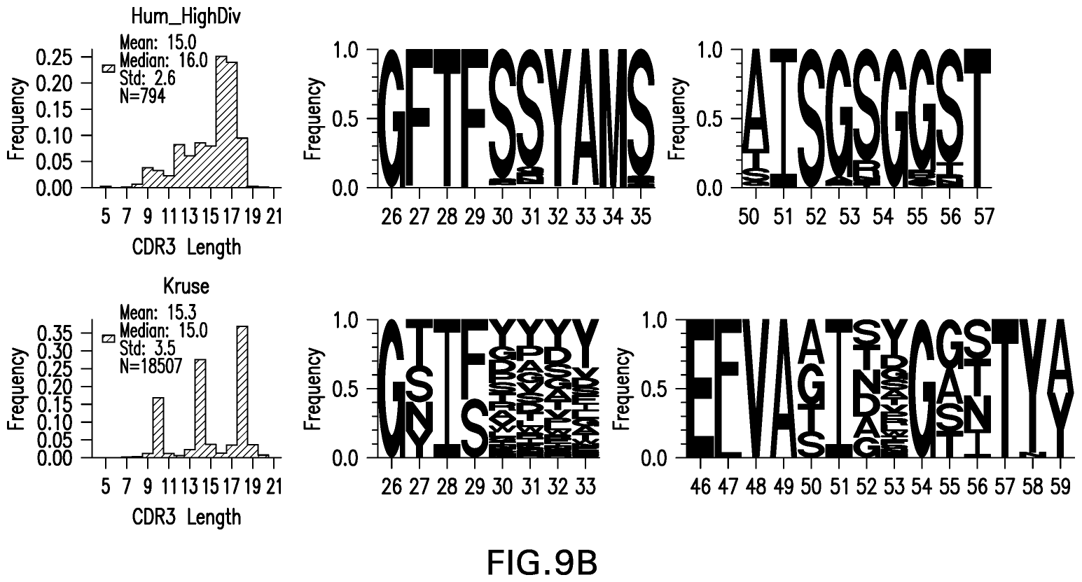

To ensure library quality, we extracted plasmid DNA from the transformed yeast and performed amplicon sequencing on the V_{H}H-encoding region (FIG. 8A and FIG. 8B). We found a distribution of CDRH3 lengths as expected. In addition, we observed that diversity was introduced correctly into the CDRH1 and CDRH2 as dictated by the design principles (FIG. 8A and FIG. 8B).

### Mouse PD-1 Campaign

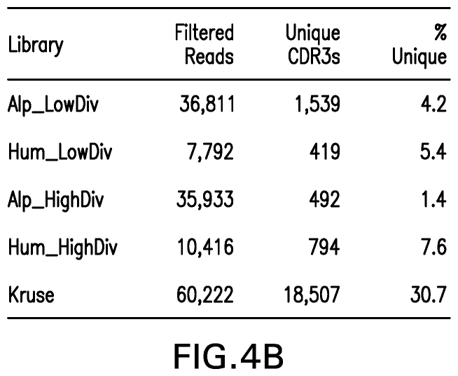

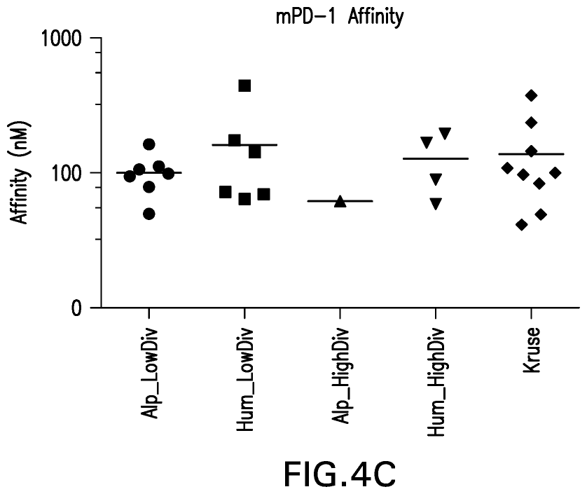

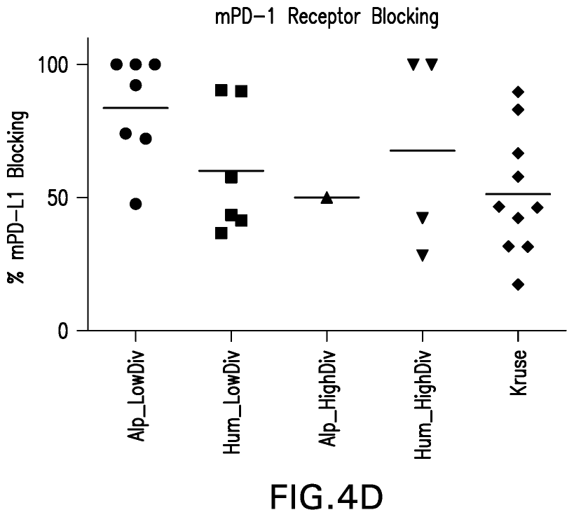

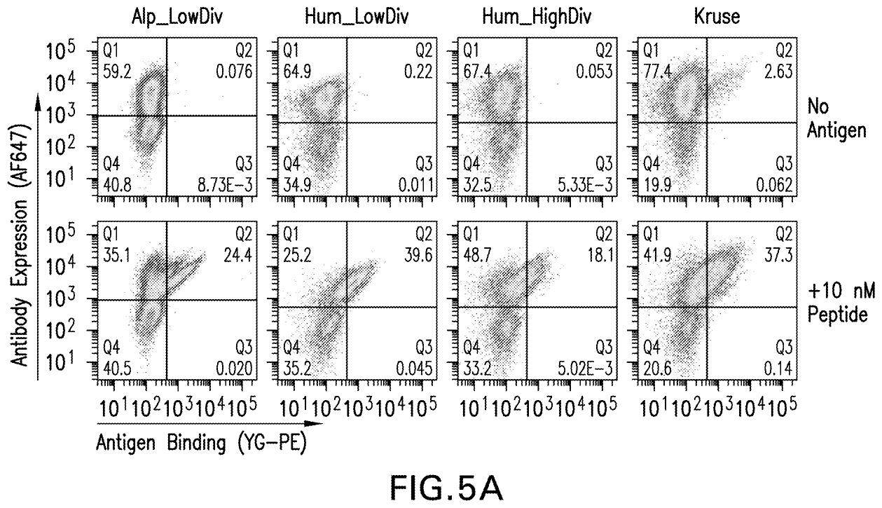

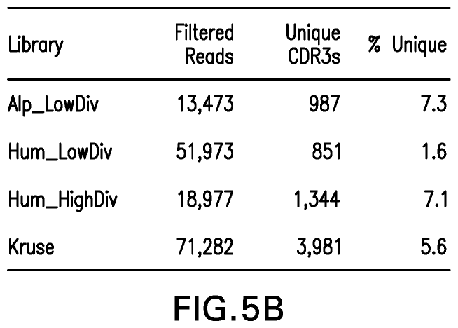

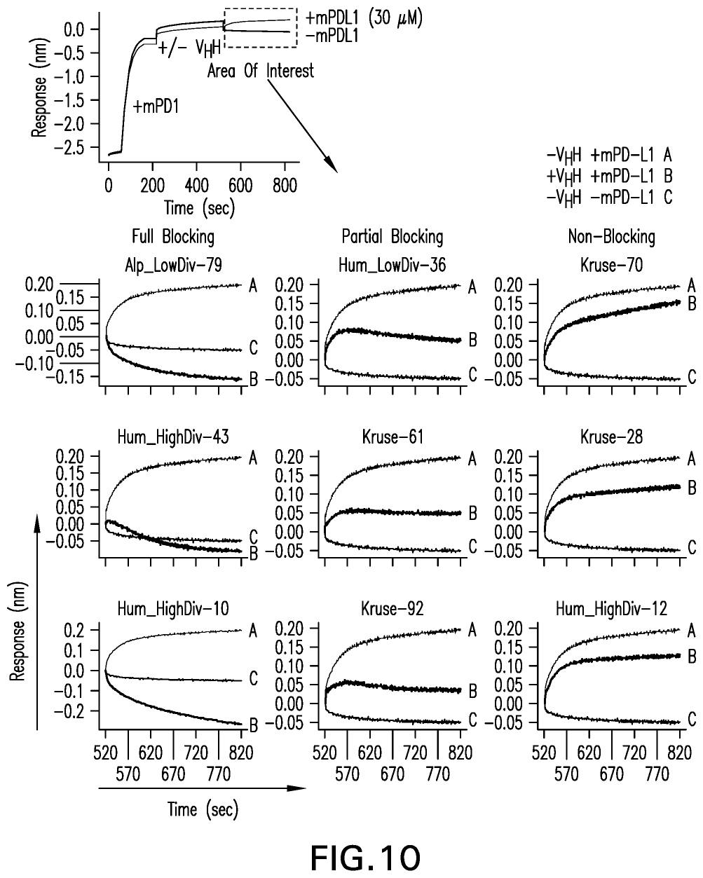

To compare performance of the libraries, we first conducted an antibody discovery campaign against the murine ortholog of programmed cell death protein 1 (mPD-1). PD-1 is involved in regulation of T cell activity (Sharpe et al., Nat. Rev. Immunol. 18, 153-167 (2018)), and PD-1 targeting monoclonal antibodies have been highly successful as therapeutic agents (Peters et al., Cancer Treat. Rev. 62, 39-49 (2018); Francisco et al., Immunol. Rev. 236, 219-242 (2010)). We first performed two rounds of magnetic cell sorting (MACS) with each of the five libraries described herein incubated with biotinylated mPD-1, followed by four rounds of fluorescent-activated cell sorting (FACS). Antigen-specific binders could be found in each for the five libraries after the fourth round of FACS, with a very low occurrence of reagent-specific binders (FIG. 4A). The clones binding to mPD-1 after the fourth round of FACS were analyzed by NGS to estimate the total clonal diversity present in the binding population. Our synthetic libraries all showed similar levels of clonal diversity, although the high diversity alpaca synthetic library (Alp_HighDiv) was heavily skewed towards a few dominant clones. The Kruse library had a higher proportion of unique clones in the enriched population than any of the other libraries (30% vs 1-7%). We also observed that longer CDR3 lengths were enriched compared to the libraries before selection (FIG. 9A and FIG. 9B). More specifically, we observed a bimodal distribution centered around 13 amino acids and 17 amino acids in our four synthetic libraries, possibly indicating two distinct modes of interaction.

We went on to produce a selected number of antigen-specific clones as recombinant proteins to measure their binding affinity and biophysical properties (see Methods in Example 2 for details on selection criteria). We expressed a total of 37 clones (22 from our four synthetic libraries and 15 from the Kruse library). Clones from each library displayed similar binding affinity profiles, with affinities ranging from 40 — 400 nM (FIG. 4C). The difference in affinities between libraries was not significant (p=0.94, Kruskal-Wallis test). The affinity range we observe here is consistent with the antigen concentrations used during MACS and FACS selection (100 nM throughout, 50 nM for final sort). Therefore, we conclude that all libraries described here can generate productive binders against mPD-1.

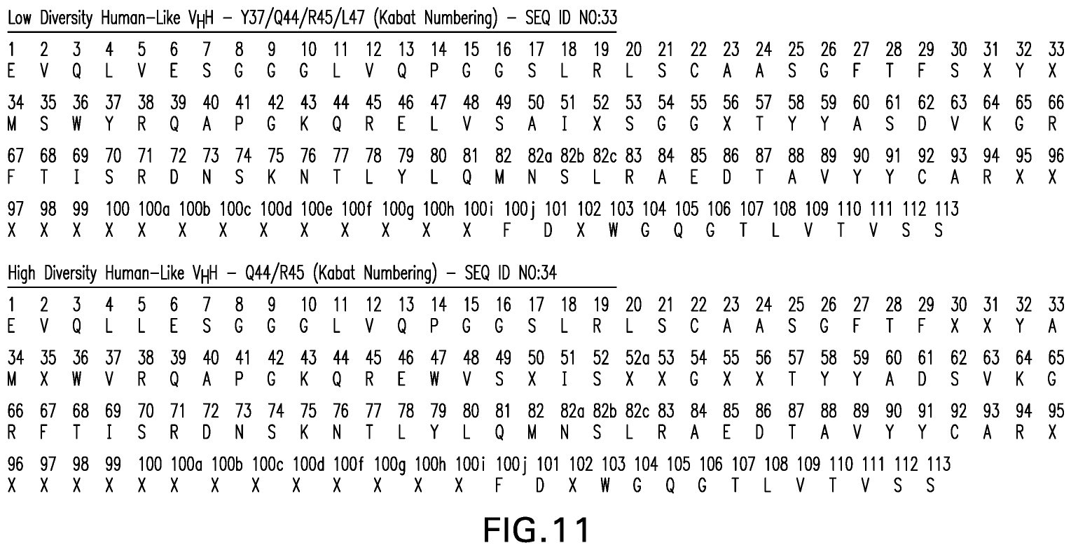

We also tested ability of the V_{H}Hs to block association of mPD-1 with its receptor, mPD-L1. This was used as a proxy to measure the number of distinct epitopes targeted by the V_{H}H clones (blocking vs. non-blocking epitopes), as well as to assess whether the libraries yielded V_{H}H that have functional activity. We used an in vitro assay to measure receptor blocking, where mPD-1 was immobilized on a biosensor, bound to a V_{H}H, then bound to mPD-L1. We were able to detect blocking activity for many of the V_{H}H using this assay (FIG. 4D; raw data in FIG. 10). Library Alp_LowDiv in particular showed a large number of clones with blocking activity. Notably, the Kruse library, although yielding clones with large sequence diversity and high affinity, generated clones with significantly less receptor blocking activity (p=0.0067 compared to Alp_LowDiv; p=0.07 compared to all our synthetic libraries; Mann-Whitney test). Therefore, we can conclude that the synthetic libraries described herein generate medium-affinity clones with functional activity in blocking receptor association.

### Peptide Campaign

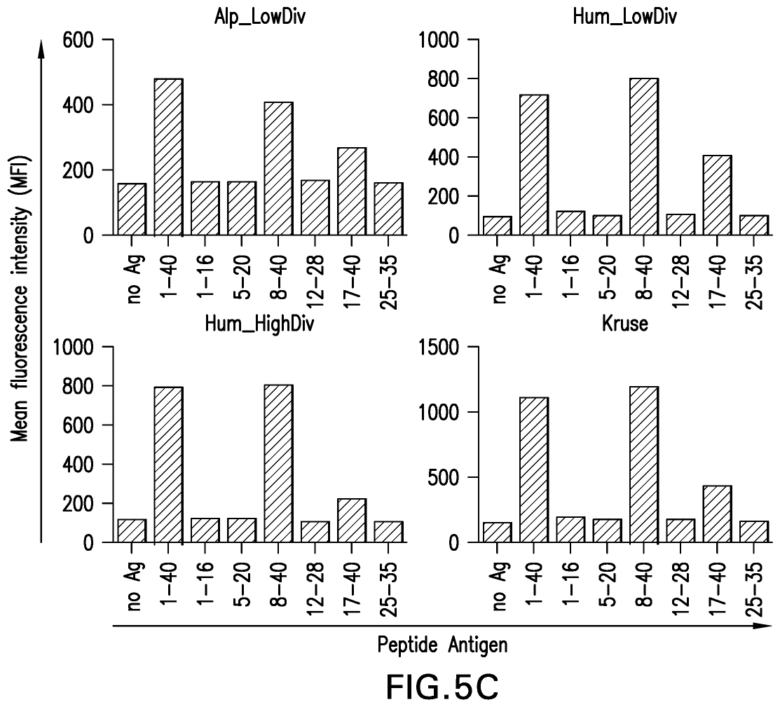

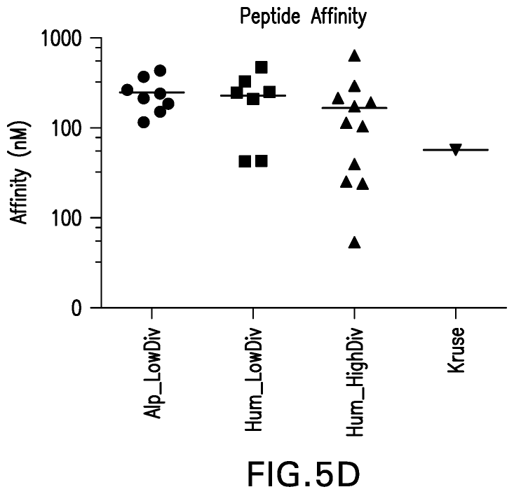

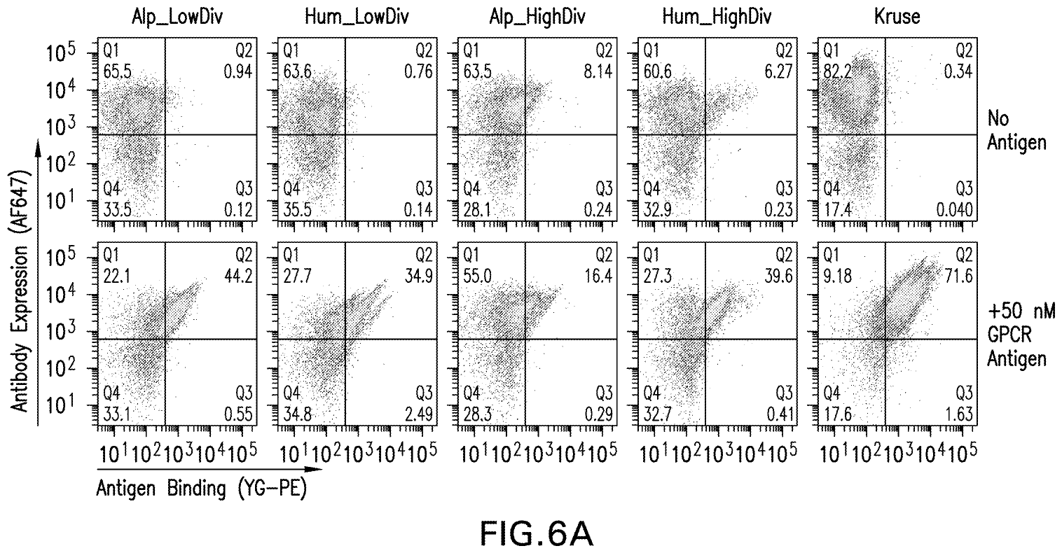

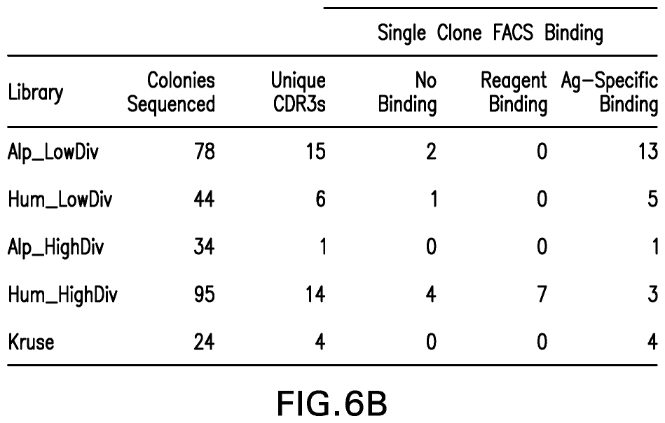

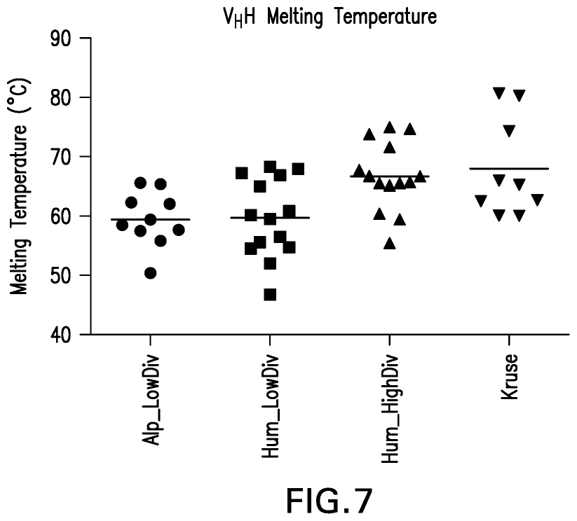

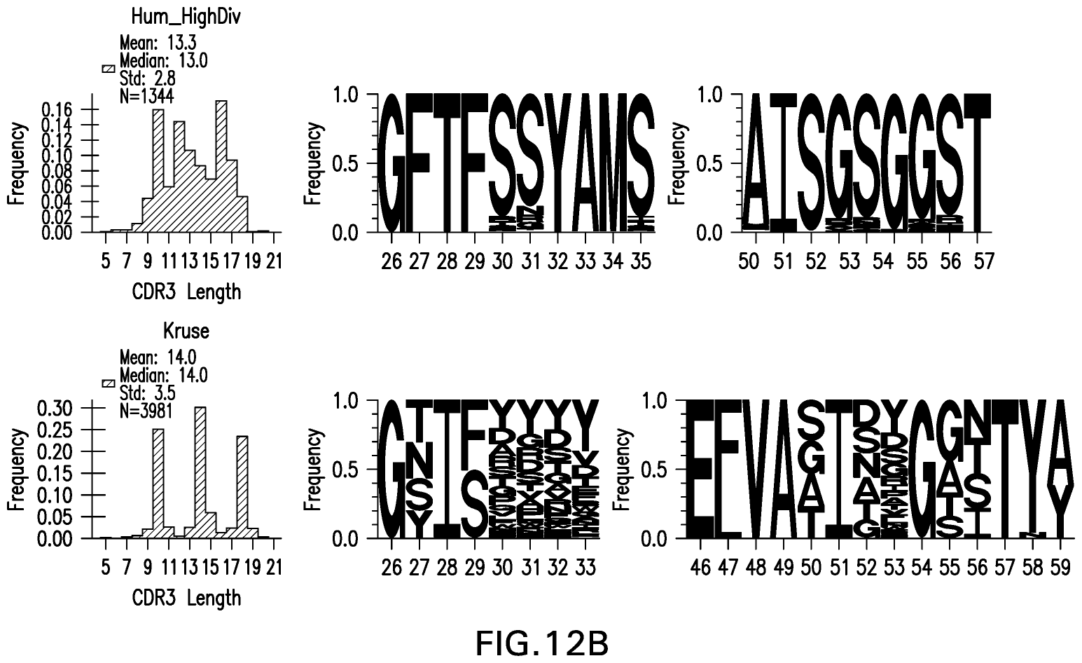

The next antibody discovery campaign was performed against a 40-amino acid Aβ peptide (“test peptide”) to assess the productivity against the peptide target. Peptide binding can be challenging for V_{H}H, since peptides frequently bind in a groove formed between the heavy and light chains of a conventional antibody (Wilson & Stanfield, Curr. Opin. Struct. Biol. 4, 857-867 (1994); Stanfield & Wilson, Curr. Opin. Struct. Biol. 5, 103-113 (1995)). As in the mPD-1 campaign, we performed two rounds of MACS and four rounds of FACS selection against biotinylated test peptide. N-terminal and C-terminal biotinylated peptides were alternated during selection to avoid enriching for clones recognizing biotin-induced conformations. After four rounds of FACS selection we observed many antigen-specific binders from four of the five libraries. Library Alp_HighDiv was observed to have only reagent-specific binders after the second round of FACS and was therefore excluded from further analysis (data not shown). NGS analysis showed a clonal diversity ranging from 1.6% unique (Hum_LowDiv) to 7.3% unique (Alp_LowDiv) in the final sorted population (FIG. 5B). The CDRH3 distribution did not show a clear skewing to longer loops (See FIG. 12A and FIG. 12B), in contrast to the long loops seen after mPD-1 selection (See FIG. 9A and FIG. 9B).

To determine which region of the test peptide was being targeted by the libraries, we incubated different overlapping peptides with the sorted library outputs and measured binding via FACS (FIG. 5C). We used a total of six overlapping peptides spanning the length of the test peptide, based on the reported binding epitopes of known mAbs against the peptide. The four libraries exhibited similar patterns of epitope recognition. The majority of clones recognized test peptide 8-40, with many of those also recognizing test peptide 17-40. Libraries Hum HighDiv and Kruse show a notable difference in binding to test peptide 8-40 vs. test peptide 17-40, indicating that there are clones targeting the internal region of the test peptide (residues 8-17). There was very little binding observed to test peptide 1-16 in any of the libraries. Overall, we conclude that all libraries produce clones targeting a variety of epitopes covering residues 8-17 and 17-40 of the test peptide, and that there are not significant difference between the libraries in their epitope coverage.

We then produced a total of 42 recombinant clones to characterize binding affinity using biolayer interferometry. As shown in FIG. 5D, we observed clear differences between the libraries in terms of their binding affinities. Library Alp_LowDiv produced clones with the weakest binding affinities, ranging from 100-400 nM. Hum_LowDiv produced a similar profile, but with two clones with affinity near 40 nM. Hum_HighDiv produced by far the best clones, with many showing sub-100 nM affinity, and one clone with an affinity of 5 nM.

Although we produced seven clones from the Kruse library, only three of the seven produced protein, and of the three, binding affinity could only be measured for one (˜50 nM). We therefore conclude that all our synthetic libraries were highly productive in generative binders against the test peptide, with Hum_HighDiv producing the highest affinity clones.

### GPCR Campaign

V_{H}H are frequently used as chaperones to induce crystal formation in difficult proteins, in particular for GPCRs (Mujić-Delić et al., Trends Pharmacol. Sci. 35, 247-255 (2014); Miao & McCammon, Proc. Natl. Acad. Sci. U. S. A. 115, 3036-3041 (2018); Rasmussen et al., Nature 469, 175-181 (2011); Wingler et al., Cell 176, 479-490.e12 (2019)). We therefore wanted to test if our libraries were suitable for obtaining V_{H}H specific to a GPCR target. We ran a discovery campaign against GPCR target MrgX1 solubilized in detergent micelles, bound to an antagonist small molecule. In contrast to previous campaigns, where reagent binders were minimized by alternating the secondary reagents used in FACS, in the GPCR campaign we observed a very high frequency of reagent binders, specifically those binding streptavidin and PE. To avoid background, we performed a preclear step, using magnetic beads to remove yeast cells that bind to streptavidin-coated beads, prior to FACS rounds 2-4. In addition, we switched from PE to a small molecule fluorophore (DyLight 550) to reduce fluorophore binders.

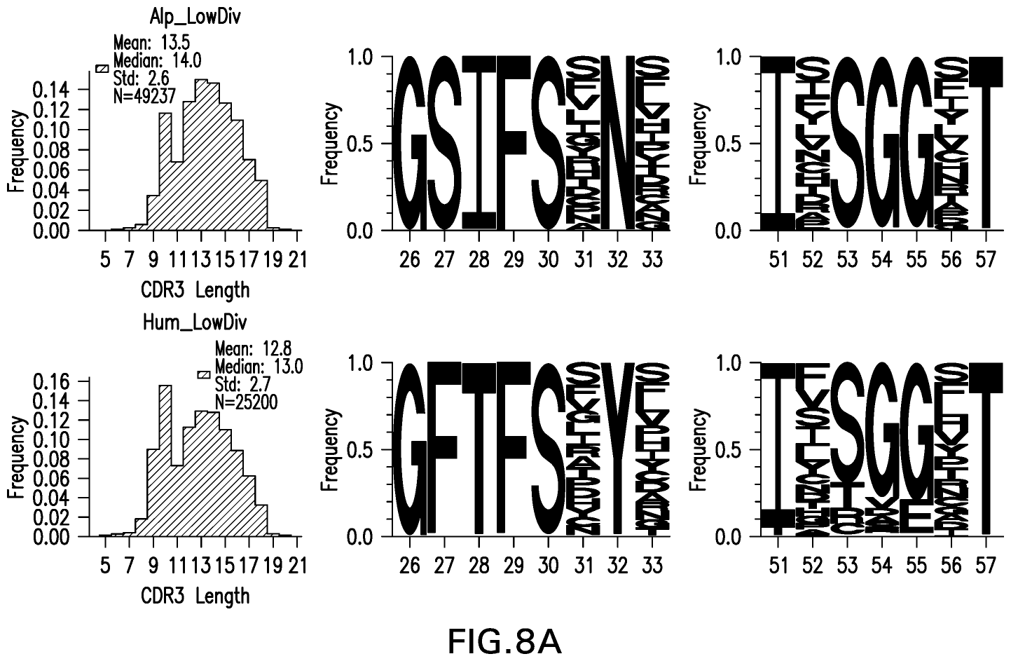

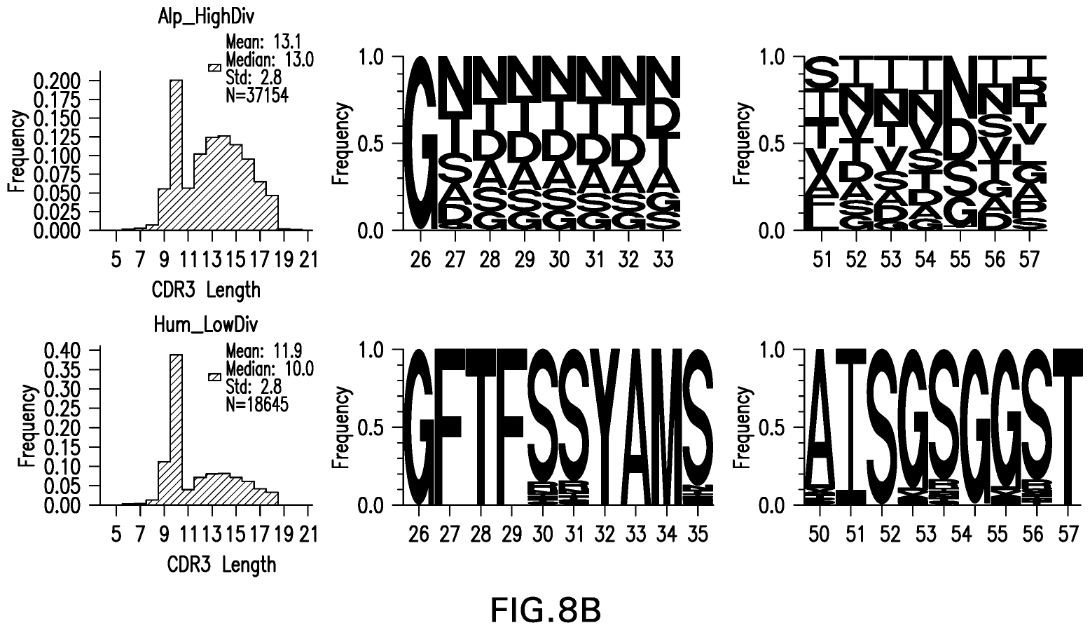

After four rounds of selection we were able to identify antigen-specific binders for all five libraries (FIG. 6A and FIG. 6B). Although the level of background binding was higher than in previous campaigns, we still observed enrichment for binding level with antigen as opposed to without.

### Biophysical Properties of V_{H}H

The goal of an antibody discovery campaign is to identify high affinity, specific antibodies targeting an antigen of interest. However, if the eventual goal is to produce a biotherapeutic, these molecules must have additional properties to be useful, such as thermal stability, high yield, and ease of production. We compared the protein production characteristics of the clones produced from the mPD-1 and test peptide campaigns (Table 4).

TABLE 4
Protein production characteristics of VHH
produced in mammalian (CHO) cells.

| | Average | | | |
|---|---|---|---|---|
| | protein | # clones that | # expressed | Overall |
| | yield | could be | clones that | conversion |
| | (mg/L) | expressed | bound | rate |
| Alp LowDiv | 26.8 | 18/21 | 15/18 | 71% |
| Hum LowDiv | 21.3 | 16/19 | 13/16 | 68% |
| Alp HighDiv | 1.6 | 1/2 | 1/1 | 50% |
| Hum HighDiv | 29.8 | 15/15 | 15/15 | 100% |
| Kruse | 22.7 | 16/22 | 11/16 | 50% |

Average yield (mg protein per liter culture) is shown as well as rate of conversion from selected sequences to binding proteins.

Four of the five libraries were very similar in the average protein yield from 30 mL mammalian cell culture, with library Alp_HighDiv an outlier in terms of poor expression. However, they differed in the overall conversion rate, defined as the number of clones that could be produced, purified, and successfully bound to the antigen divided by the total clones attempted. Whereas all the clones from library Hum_HighDiv produced protein that bound to the antigen of interest, the Kruse library was not as productive, with only 50% of clones making it through this process. Therefore, we conclude that the Merck libraries produce well-behaved clones capable of expression as recombinant protein.

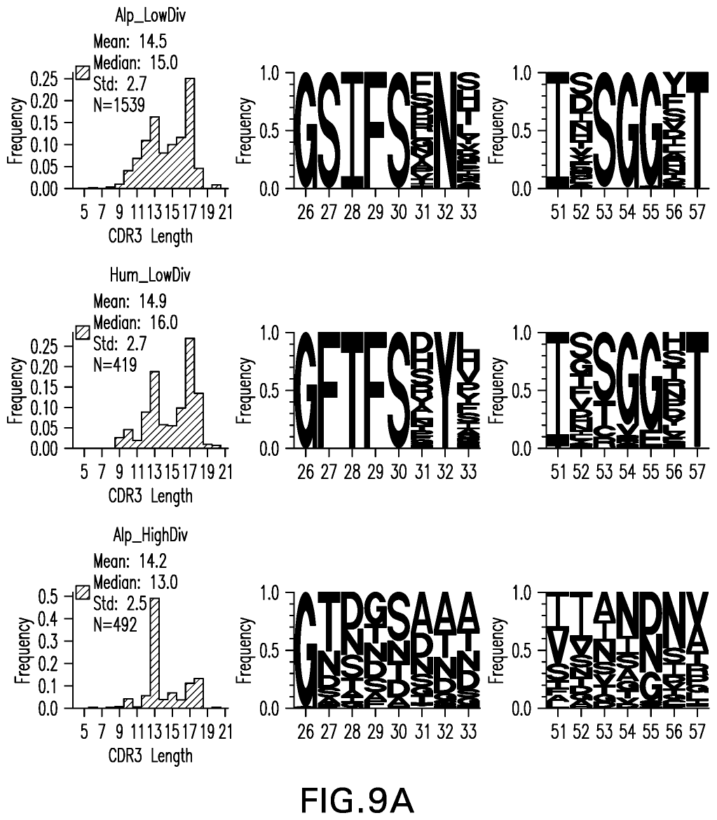

In addition, we measured the thermal stability of the recombinant V_{H}H using differential scanning fluorimetry (DSF). Since fully alpaca, humanized, and consensus alpaca frameworks were used to build the various libraries, we hypothesized that this may have an impact on the thermal stability of the recombinant proteins. FIG. 7 shows that the choice of framework had little impact on the melting temperature (Tm). In particular, we were interested in the difference between the libraries Alp_LowDiv and Hum_LowDiv, since these were identical except for the use of fully alpaca or partially humanized frameworks, respectively. We observed little difference in Tm between these two libraries, indicating that partial humanization did not negatively impact thermal stability of the molecules. The highest melting temperatures were exhibited by clones from the Kruse and Hum_HighDiv libraries, with Tms up to 80° C. exhibiting by V_{H}H from the Kruse library. In general, we conclude that all libraries are able to generate thermostable V_{H}H that can be expressed with high yield in mammalian cell culture.

### Discussion

In this Example, we describe the construction and validation of four structure- and sequence-based V_{H}H libraries. We show that these libraries produce V_{H}H with affinity and functional characteristics comparable to, and in the case of mPD-1 receptor blocking superior to that of V_{H}H from the Kruse library, the standard in the field. The libraries were tested against three classes of protein antigens, indicating that they are general purpose in nature and can be applied to any antigen of interest with a high probability of yielding binding clones.

This work is novel in that we used a highly quantitative approach to determining how to best introduce diversity in the CDRH1 and CDRH2 regions. We used structural modeling of the known V_{H}H-antigen complexes available in the PDB to determine which residues typically contribute most strongly to binding. Not surprisingly, the contribution to binding is not evenly distributed along the CDRH1 and CDRH2 loops, and there is a strong preference for some residues to interact with antigen while others contribute more to internal stability. The energetic contributions predicted by structural modeling agree well with sequence variability in NGS datasets, giving an orthogonal indicator that the modeling predictions are sound. The analysis of V_{H}H binding characteristics presented in this study can also be used in the future to build libraries tailor-made for a given type of antigen. Here we analyzed all V_{H}H-antigen complexes to create general-purpose libraries. However, a similar analysis could be performed for a specific type of antigen to make tailored libraries.

One key question in constructing our libraries was, how much CDRH1 and CDRH2 diversity is truly necessary to generate productive binders. Alternate approaches such as the Kruse library incorporate a high degree of diversity (2.3×10¹⁰ theoretical diversity) in these loops using trinucleotide cassettes (McMahon et al., Nat. Struct. Mol. Biol. 25, 289-296 (2018)). To test if this level of diversity is necessary, we were able to directly compare the Alp_LowDiv and Alp_HighDiv libraries, which were identical except for CDRH1 and CDRH2 diversity. Not only was the extra diversity not necessary for productivity, the low diversity library performed significantly better, in terms of number of unique binders yielded and final affinity values. One potential explanation is that the high diversity library sacrificed a large proportion of clones in terms of their ability to fold properly. However, this is not borne out by our data, as the Alp_HighDiv naive library induction levels are actually superior to Alp_LowDiv. The purpose of Alp_LowDiv was to alter only positions likely to interact with antigen based on a structural rationale-based on its performance verses Alp_HighDiv, we conclude that this structural approach was successful.

Another benefit of our libraries is the fact that we used partially humanized frameworks (human-like), which are only two amino acids different from fully human frameworks. We were initially concerned about the effect that humanization may have on productivity or thermal stability of the libraries, since they are non-natural molecules. However, the humanized libraries perform as well or better than the alpaca libraries in terms of both productivity in generating binders, and thermal stability. Humanization is a common problem in the antibody discovery process, as non-human residues are frequently required for antibody affinity and stability (Ahmadzadeh et al., Monoclon. Antib. Immunodiagn. Immunother. 33, 67-73 (2014); Hwang et al., Methods 36, 35-42 (2005); Tan et al., J. Immunol. 169, 1119-1125 (2002); Mader & Kunert, PLoS One 7, 1-8 (2012)). Many approaches to antibody humanization exist; however, it is inevitable that some clones are lost due to inactivity after humanization. Our libraries Hum_LowDiv and Hum_HighDiv avoid this problem by eliminating the need for humanization after selection, without any noticeable cost in antibody fitness.

Our data is in agreement with other work done in the field regarding the binding proclivities of V_{H}H. Other synthetic libraries have been built based on structural principles. McMahon, et al. (op. cit.) used a set of 93 V_{H}H from the PDB to inspire their choices in CDRH3 lengths as well as positional variation in CDRH1 and CDRH2. Zimmermann et al. (Elife 7, e34317 (2018)) built synthetic V_{H}H libraries based on geometry of the paratope, either concave, convex, or flat. Moutel et al. (Elife 5, 1-31 (2016)) and Yan et al. (J. Transl. Med. 12, 1-12 (2014)) have also presented synthetic V_{H}H libraries using phage display, which were successful in antibody identification campaigns although without the structural guidance presented in this and other work. The libraries described herein, therefore, represent a complementary approach to those that have been described in the past.

### EXAMPLE 2

This example includes the methods that were used to obtain the results disclosed in Example 1.

### Structural Analysis

To determine the structural variation in naturally occurring V_{H}H, we used a dataset of V_{H}H-antigen co-complexes from the Protein DataBank (PDB; rcsb.org). Annotated structures were downloaded from the Structural Antibody Database (SAbDab; Dunbar et al., Nucleic Acids Res. 42, D1140-6 (2014)). The filtered set of structures consisted of all unique V_{H}H-antigen complexes with protein or peptide antigens and a resolution of <3.5 Å. The structures were downloaded and manually processed to remove water and non-protein residues and renumbered starting from residue 1. Binding energies of the V_{H}H-antigen complexes were estimated using the Rosetta molecular modeling suite, version 3.819,41. Each complex was refined using Rosetta relax with constraints to the starting coordinates to prevent the backbone from making substantial movements. Constraints were placed on all Cα atoms with a standard deviation of 1.0 Å. Binding energy per residue was calculated using a custom RosettaScripts XIVIL protocol (Fleishman et al., RosettaScripts: a scripting language interface to the Rosetta macromolecular modeling suite. 6, e20161 (2011)) using the REF2015 score function19. Position of CDR loops was defined using the IMGT/DomainGapAlign tool (Lo & Lefranc, Antib. Eng. 33, 27-50 (2004)). Binding energy (AAG) and fractional binding energy (ΔΔGfractional) of each V_{H}H region were calculated as follows:

ΔΔGₜₒₜₐₗ=E_{complex}−E_{V}_{_{H}}_{H}−E_{Ag}

ΔΔG_{fractional}=ΔΔG_{region}/ΔΔGₜₒₜₐₗ

### Sequence Analysis

We downloaded two publicly available datasets of antibody repertoires from alpaca (Lama pacos) and Bactrian camel (Camelus bactrianus) from the NCBI Sequence Read Archive44 (SRA, codes DRR01858222 and SRR354421723, respectively). We downloaded the raw FASTQ files using the fastq-dump function from the SRA toolkit (Leinonen et al., Nucleic Acids Res. 39, 2010-2012 (2011)) and assembled the paired end reads using PANDAseq (Masella et al., BMC Bioinformatics 13, 559; author reply 559-60 (2012)). Germline genes were assigned using IgBLAST (Ye et al., Nucleic Acids Res. 41, 34-40 (2013)) version L9.0, using a custom database of Vicugna pacos genes from the IMGT reference database (Lo et al. (op. cit.). Reads were filtered by the following criteria: 1) successful V and J gene assignment, with an E value cutoff of 10-4, 2) CDRH1, 2, and 3 able to be assigned, and 3) no stop codon in translated amino acid sequence (in the case of sorted outputs). Data were deduplicated by CDRH3. Sequence profiles of CDRH1 and CDRH2 amino acids were generated using the WebLogo tool (Crooks et al., Genome Res. 14, 1188-1190 (2004)). Plots were created in Python using the Matplotlib library (Hunter et al., Comput. Sci. Eng. 9, 99-104 (2007)).

### Library Design

Using structural and sequence constraints, four V_{H}H libraries were designed based on fully V_{H}H and partially humanized frameworks. Humanization was done based on alignment of the V_{H}H framework to the closest human germline IGHV gene using the IMGT reference database (Lefranc, Cold Spring Harb. Protoc. 6, 595-603 (2011)). Based on structural and sequence analysis two positions in the CDRH1 and CDRH2 (four positions total) were diversified in libraries Alp_LowDiv and Hum_LowDiv. Library Alp_HighDiv was diversified in 14 positions total (seven in CDRH1 and seven in CDRH2), using a reduced codon vocabulary to incorporate the amino acids most commonly observed in the NGS datasets, on a positional basis. Library Hum_HighDiv used spiked nucleotide ratios of 79:7:7:7 to maintain a proportion of 49% germline codon. Libraries were synthesized using GeneArt DNA synthesis (Thermo Fisher Scientific).

A common CDRH3 library was designed and fused to the framework of each library. The CDRH3 fragments were synthesized using trinucleotide mutagenesis (TRIM) to control amino acid composition (see for example, Shim, BMB Reps. 48:489-494 (2015); Knappik et al., J. Mol. Biol. 296: 57-86 (2000); GeneArt of Thermo Fisher Scientific).

### Library Construction and Quality Control (QC)

To construct the four libraries, genes encoding the DNA sequence of the IGHV-gene encoded region of the antibody were synthesized (Thermo Fisher Scientific), with a 5′ region conferring a 200 bp overlap with the destination vector. The full antibody gene was assembled using a three-step PCR overlap extension. First, a 3′ recombination arm of the destination vector was amplified with an HA tag inserted directly downstream of the CDRH3 region, conferring an overlap of 410 bp with the destination vector. Next the 3′ recombination arm was fused to the CDRH3 fragments using PCR overlap extension. Lastly, the IGHV-gene encoded fragment was assembled with the CDRH3-3′ overlap fragment using PCR overlap extension. Care was taken to ensure that at least 10¹¹ molecules of library DNA fragments were included in each step of overlap extension to ensure that diversity was not lost. Fully assembled fragments were blunt-end cloned into the pJET1.2 vector using the CloneJet cloning kit (ThermoFisher) and 100 clones per library were sequenced to ensure library quality before yeast transformation.

### Yeast Transformation

Yeast libraries were generated by high-efficiency transformation of a genetically modified version of the BJ5465 strain (ATCC). Cells were grown to an OD of 1.6, spun down and washed 2× with water (or, in certain cases, 1 M sorbitol) and 1× with electroporation buffer (1 M sorbitol+1 mM CaCl₂). Cells were then incubated in pre-treatment buffer (0.1 M LiAc+2.5 mM TCEP) shaking for 30 minutes at 30° C. Next, cells were spun down and wash 3× with cold electroporation buffer. Cells were then resuspended in electroporation buffer to a final concentration of 2×10⁹ cells/mL. 4 μg linearized vector and 12 μg insert were added to 400 μL cells per cuvette. Electroporation using the exponential decay protocol was performed with a 2 mm cuvette with the following parameters: 2.75 kV, 200Ω resistance. 25 uF capacitance, typically resulting in a time constant of 3.5-4.0 ms. After electroporation, recovery media (equal parts YPD media and 1 M sorbitol) was added and cells were incubated shaking for 1 hour at 30° C. Cells were then spun down and resuspended in 1 M sorbitol at dilutions of 10⁻⁶, 10⁻⁷, and 10⁻⁸, and plated on glucose dropout media lacking leucine. Colonies were counted after three days growth to measure number of transformants.

### Next-Generation Sequencing (NGS) and Analysis

Library characteristics after transformation and selection were assessed by next-generation sequencing. Roughly 5×10⁸ cells were spun down from each transformed library, plasmid DNA was extracted, and the V_{H}H-encoding region was amplified by PCR. The amplified fragments were sequenced using Illumina MiSeq 2×250 amplicon sequencing (GeneWiz). Forward and reverse reads were assembled using PANDASEQ45 and germline genes and CDR loops were assigned using IgBLAST46. Reads were filtered using the same criteria as previously described.

### Display and Induction

To induce antibody expression on the yeast surface, cells were first grown in 4% glucose dropout media lacking leucine overnight at 30° C. Cells were then switched to 4% raffinose media at a starting OD of 1.0 to derepress the GAL1 promoter and grown overnight at 30° C. The following morning, cells were switched to induction media (dropout media containing 2% raffinose and 2% galactose) to induce expression of V_{H}H under control of the GAL1 promoter. Induction media was supplemented with doxycycline at a final concentration of 22.5 μM and an O-linked glycosylation inhibitor (Argyros, et al., PLoS One doi:10.1371/journal.pone.0062229 (2013)) at a final concentration of 1.8 mg/L.

### Magnetic Sorting (MACS)

To isolate antigen-specific V_{H}H, libraries underwent two rounds of MACS followed by four rounds of fluorescence-activated cell sorting (FACS). For each library, 10¹⁰ cells from frozen transformation stocks were thawed and grown in 1 L selective media, and expression was induced as previously described. Induction level of each library before MACS was confirmed by flow cytometry. 3×10¹⁰ induced cells per library were spun down and washed 3× with PBS-F (PBS+0.1% bovine serum albumin). Cells were then labeled with 100 nM antigen in 20 mL PBS-F for 1 hour shaking at 30° C. After labeling, cells were spun down and washed 3× with cold PBS-F, then incubated with 500 μL streptavidin microbeads (Miltenyi Biotec) in 40 mL PBS-F for 30 minutes with rotation at 4° C. Antigen-bound cells were isolated by passing through an LS column (Miltenyi), washing 3× with 3 mL PBS-F. Cells were then eluted with 5 mL selective media and grown overnight. A subsequent round of magnetic sorting was performed, starting with 5×10⁹ induced cells per library. The second round of magnetic sorting was done following the previously described protocol, with the following modifications: 1) total volume during antigen incubation step was adjusted to 2 mL, 2) total volume during microbead incubation step was adjusted to 5 mL, and 3) anti-biotin microbeads were used to avoid enriching for streptavidin-specific binders.

### FACS

After library sizes were reduced by magnetic sorting, FACS was used to identify antigen-specific V_{H}H. 5×10⁸ cells per library were passaged and induced and 10⁹ induced cells were spun down and washed 3× with PBS-F. Cells were incubated with 100 nM antigen in a total volume of 1 mL for 1 hour at 30° C. shaking, then washed again 3× with PBS-F. Next, cells were incubated with three secondary antibodies: an anti-HA tag mouse monoclonal antibody conjugated to AlexaFluor 647 (Thermo Fisher Scientific) to detect V_{H}H expression, neutravidin conjugated to PE (Thermo Fisher Scientific) to detect antigen binding, and YOYO1 nuclear dye (Thermo Fisher Scientific) to measure cell viability. The secondary antibodies were added at a dilution of 1:1000, 1:200, and 1:2000, respectively, in a total volume of 10 mL, and incubated for 30 minutes on ice. After secondary incubation, cells were washed 3× with PBS-F and diluted in PBS-F for FACS screening. All FACS sorting was done on an Aria III flow cytometer (BD Biosciences). Gates were drawn to include a single population in an FSC/SSC plot and to exclude doublets on an FSC-A/FSC-H plot. In addition the FITC-negative population was gated to remove YOYO1-stained dead cells. For the GPCR campaign, PBS-F buffer was supplemented with detergent (0.05% dodecylmaltoside, 0.005% cholesteryl hemisuccinate) in all MACS and FACS stages. In addition, the primary incubation was performed in the presence of 20 μM antagonist. A preclear step was included in this campaign by incubating cells with 250 μL streptavidin beads at room temperature rocking for 30 minutes and passed through an LD column (Miltenyi). Flow-through cells were then subjected to FACS labeling as described above.

Cells positive in both PE and AlexaFluor 647 channels were sorted into selective media, grown overnight, and passaged for a subsequent round of enrichment. The last round of selection was performed with an antigen concentration ranging from 10-50 nM to isolate high affinity binders. The secondary antibody for antigen detection was alternated between neutravidin-PE and streptavidin-DyLight 550 (Thermo Fisher Scientific) to reduce reagent-specific binders. In the test peptide campaign, N- and C-terminal biotin-linked test peptides were alternated during FACS rounds to reduce biotin-specific binders.

After four rounds of selection, single clones were isolated and subsequently grown and induced in a plate format. Cells were sequenced by colony PCR, and single clone binding in plate format was confirmed by screening against 100 nM antigen on a Canto II flow cytometer (BD Biosciences). From each plate, clones with a unique CDRH3 sequence that displayed binding in single-cell format were selected for recombinant production.

### Recombinant Production

The V_{H}H-encoding region of selected clones was amplified and subcloned into the pTT5 mammalian expression vector, flanked by a penta-His tag. Recombinant V_{H}H were expressed by transient transfection of 30 mL cultures of ExpiCHO-S cells (Thermo Fisher Scientific) following the recommended protocol. Supernatants were harvested after seven days and filter-sterilized with a 0.2-μm filter. Supernatant was bound to Amsphere A3 Protein A resin (JSR Life Sciences) in a batch format, with 500 μL resin per sample, and purified using a gravity column. The resin was washed with 10 column volumes (CV) PBS and eluted with 4 CV elution buffer (0.5 M glycine, pH 3.5) before the addition of 140 μL neutralization buffer (1 M Tris, pH 8) to result in a final pH of 4.8-5.0.

### Antigen Generation

mPD-1

Expression construct encoding the extracellular domains of murine PD-1 (from Leu-25 to Glu-150 with the unpaired Cys-83 mutated to Ser) was designed. The gene was constructed as soluble monomer with a 6×-His tag at the C-terminus. The sequence was codon optimized for expression in Chinese hamster ovary (CHO) cells and synthesized. Synthesized gene was cloned into the pTT5 mammalian expression vector. The protein was expressed by transient transfection of Expi293 cells (Thermo Fisher Scientific). The harvested supernatant was filter-sterilized with a 0.2-μm filter and purified using affinity chromatography (GE Nickel Excel column). After purification, the protein was further polished with size exclusion chromatography (GE Healthcare SOURCE 15Q column).

### Test Peptide

Test peptide Aβ was synthesized by Genscript with either a N-terminal biotin or C-terminal lysine-linked biotin, at a purity of >90%. In both cases the biotin moiety was separated from the test peptide by a polyethylene glycol (PEG) 6 linker on either the N- or C-terminus, respectively. In addition, peptides spanning residues 1-16, 5-20, 8-40, 12-28, 17-40, or 25-35 were synthesized to perform epitope mapping, with a N-terminal biotin and 90% purity.

### Construct Engineering, Expression and Purification of GCPR

The GPCR MrgX1 construct used for screening lacked the first 5 N-terminal and last 19 C-terminal residues. To boost expression and to stabilize the inactive state, a Gly to Arg mutation at position 3.41 (Ballesteros-Weinstein (BW) numbering) and C to A mutation at position 3.51 were introduced. The construct also contained a haemagglutinin (HA) signal sequence followed by a FLAG tag at the N-terminus and an Avi-tag and a 10× His tag at the C-terminus to enable purification by metal affinity chromatography and labeling with biotin.

### Construct was Synthesized by Genescript.

High-titer recombinant baculovirus was generated in Sf21 cells using BestBAC Linearized DNA v-cath/chitinase deletion (Expression Systems) according to the Titerless Infected-Cells Preservation and Scale-Up (TIPS) Method (Wasilko & Lee, Bioprocess. J. 5: 29-32 (2006)). GPCR antigen was expressed in Sf21 cells infected at a density of 2-3×10⁶ cells per mL in SF-900 II media (Invitrogen) and an MOI of 3 for 72 hours.

Cells were harvested by centrifugation at 72 hours post-infection and stored at −80° C. until use. Frozen cells were resuspended in a low-salt buffer containing 10 mM HEPES, pH 7.5, 10 mM MgCl₂, 20 mM KCl and Roche EDTA-free cOmplete protease inhibitor cocktail tablets. Membrane fractions were isolated from 5 L of biomass by repeated Dounce homogenization and ultracentrifugation, once in low-salt buffer and once in high-salt buffer (10 mM HEPES pH 7.5, 10 mM MgCl₂, 20 mM KCl, 1 M NaCl, and protease inhibitor cocktail tablets). Membranes were stored at −80° C. until use.

Frozen membranes were thawed and resuspended in 40 mM Tris pH 8.0, 0.15 M NaCl, 25 μM antagonist, 1% (w/v) n-dodecyl-β-d-maltopyranoside (DDM, Anatrace)/0.1% Cholesterol hemisuccinate (CHS, Sigma-Aldrich) and Roche EDTA-free cOmplete protease inhibitor cocktail tablets. Membranes were stirred in the buffer for 1 hour at 4° C. to allow binding of the compound to the receptor, after which 1% DDM/0.01% CHS was added from a 10× stock to solubilize the membranes. Membranes were stirred for a further two hours at 4° C. to complete solubilization. Final solubilization volume was 390 ml. Insoluble fraction was removed by ultracentrifugation at 138,000 g for 30 minutes.

The supernatant was then loaded on a pre-packed 5 mL HisTrap Crude FF column (Qiagen #30410) pre-equilibrated with buffer A (40 mM Tris pH 8.0, 0.15 M NaCl, 20 μM antagonist, 0.05% (w/v) DDM/0.005% CHS) using an AKTA purifier system at flow rate of 2 mL/minute. The sample was washed with about 20 CVs of buffer A containing 65 mM imidazole (BioUltra, Sigma-Aldrich) and eluted with 250 mM in a single 9 mL fraction. To prepare the sample for biotinylation, a buffer exchange into buffer F (10 mM Tris pH 8.0, 0.15 M NaCl, 20 μM antagonist, 0.05% (w/v) DDM/0.005% CHS) was performed using PD10 columns (GE Healthcare) to removed imidazole and the protein was concentrated to 1.8 mg/mL as measured using a Nanodrop. Biotinylation reaction was set up using the BirA-500 kit (Avidity LLC) and allowed to proceed overnight at 4° C.

The overnight sample was subsequently concentrated to about 1 mL using an Amicon Ultra −15 Centrifugal filter with 100 kDa molecular weight cutoff (Millipore) and subjected to an ultracentrifuge spin at 250,000 g for 20 minutes. The concentrated sample was split into 2×500 μL aliquots and purified on a Superdex 200 increase 10/300 GL gel filtration column (GE Healthcare). Completion of biotinylation was verified in a gel-shift assay using streptavidin.

### Affinity Determination

Binding affinity was measured using Biolayer Interferometry (BLI) with a FortéBio Octet HTX instrument. Biotinylated antigen was loaded onto streptavidin biosensors at a concentration of 100 nM in kinetics buffer (PBS+0.1% BSA). The binding experiments were performed with the following steps: 1) baseline in kinetics buffer for 30 seconds, 2) loading of antigen for 180 seconds, to achieve a loading response of at least 1 nm, 3) baseline for 60 seconds, 4) association of 1 μM V_{H}H for 300 seconds, and 5) dissociation into kinetics buffer for 180 seconds. Curves were fit to a 1:1 binding model using the FortéBio software. A negative control was included in all plates, which was untransfected mammalian cells subjected to the same purification process, to account for the effect of any carryover protein contaminants from cell culture.

In vitro receptor blocking was performed using BLI on the Octet HTX, with the following steps: 1) baseline in kinetics buffer, 2) loading of mPD-1 to streptavidin biosensors at 100 nM for 90 seconds, 3) baseline, 4) binding to 1 μM V_{H}H for 300 seconds, 5) binding to mPD-L1 at 30 μM for 300 seconds. The response after binding to mPD-L1 was normalized compared to a positive control where no V_{H}H was added, and a negative control where no V_{H}H and no mPD-L1 was added, to calculate the percent receptor blocking. In several cases the response after mPD-L1 was lower than the negative control, due to the impact of V_{H}H dissociating from the biosensor—these samples were treated as 100% blocking.

### Differential Scanning Fluorimetry

Melting temperatures were measured using Differential Scanning Fluorimetry (DSF) on a Prometheus NT.Plex instrument (NanoTemper Technologies). Protein unfolding was monitored by intrinsic fluorescence, as measured by fluorescence intensity ratio at 350/330 nm. Proteins were loaded onto the capillaries at concentrations ranging from 6-60 μM. A temperature scan from 20° C. to 95° C. was performed at a rate of 1° C./minute. First derivative plots were used to determine the melting temperature (Tm).

TABLE OF SEQUENCES

| SEQ | | |
|---|---|---|
| ID | | |
| NO: | Description | Sequence |
| 1 | Low diversity human- | EVQLVESGGGLVQPGGSLRLSCAASGFTFSXYXMSWVR |
| | like V_{H}H comprising | QAPGKQREWVSAIXSGGXTYYADSVKGRFTISRDNSKN |
| | GHV3-23*04 VH with | TLYLQMNSLRAEDTAVYYCARXXXXXXXXXXXXXXXF |
| | Q44/R45, wherein each | DXWGQGTLVTVSS |
| | X is independently any | |
| | amino acid except C; | |
| | wherein following | |
| | CAR each X beginning | |
| | with X4 and ending | |
| | with X15 may be | |
| | present or absent | |
| 2 | Low diversity human- | EVQLVESGGGLVQPGGSLRLSCAASGFTFSXYXMSWYR |
| | like V_{H}H comprising | QAPGKQRELVSAIXSGGXTYYADSVKGRFTISRDNSKNT |
| | GHV3-23*04 VH with | LYLQMNSLRAEDTAVYYCARXXXXXXXXXXXXXXXF |
| | Y37Q44/R45/L47, | DXWGQGTLVTVSS |
| | wherein each X is | |
| | independently any | |
| | amino acid except C; | |
| | wherein following | |
| | CAR each X beginning | |
| | with X4 and ending | |
| | with X15 may be | |
| | present or absent | |
| 3 | High diversity human- | EVQLLESGGGLVQPGGSLRLSCAASGFTFXXYAMXWVR |
| | like V_{H}H comprising | QAPGKQREWVSXISXXGXXTYYADSVKGRFTISRDNSK |
| | GHV3-23*04 VH with | NTLYLQMNSLRAEDTAVYYCARXXXXXXXXXXXXXX |
| | Q44/R45, wherein each | XFDXWGQGTLVTVSS |
| | X is independently any | |
| | amino acid except C; | |
| | wherein following | |
| | CAR each X beginning | |
| | with X4 and ending | |
| | with X15 may be | |
| | present or absent | |
| 4 | High diversity human- | EVQLLESGGGLVQPGGSLRLSCAASGFTFXXYAMXWYR |
| | like V_{H}H comprising | QAPGKQRELVSXISXXGXXTYYADSVKGRFTISRDNSKN |
| | GHV3-23*04 VH with | TLYLQMNSLRAEDTAVYYCARXXXXXXXXXXXXXXXF |
| | Y37/Q44/R45/L47, | DXWGQGTLVTVSS |
| | wherein each X is | |
| | independently any | |
| | amino acid except C; | |
| | wherein following | |
| | CAR each X beginning | |
| | with X4 and ending | |
| | with X15 may be | |
| | present or absent | |
| 5 | Low diversity alpaca | QVQLVESGGGLVQPGGSLRLSCAASGSIFSXNXMGWYR |
| | V_{H}H IGHV3S53 | QAPGKQRELVAAIXSGGXTNYADSVKGRFTISRDNAKN |
| | V_{H}H, wherein each X | TVYLQMNSLKPEDTAVYYCARXXXXXXXXXXXXXXXF |
| | is independently any | DXWGQGTLVTVSS |
| | amino acid except C; | |
| | wherein following | |
| | CAR each X beginning | |
| | with X4 and ending | |
| | with X15 may be | |
| | present or absent | |
| 6 | High diversity alpaca | QVQLVESGGGLVQPGGSLRLSCAASGXXXXXXXMGWY |
| | V_{H}H IGHV3S53 | RQAPGKQRELVAAXXXXXXXNYADSVKGRFTISRDNA |
| | V_{H}H, wherein each X | KNTVYLQMNSLKPEDTAVYYCARXXXXXXXXXXXXX |
| | is independently any | XXFDXWGQGTLVTVSS |
| | amino acid except C; | |
| | wherein following | |
| | CAR each X beginning | |
| | with X4 and ending | |
| | with X15 may be | |
| | present or absent | |
| 7 | Amino acids 1-97 of | QVQLVESGGGLVQPGGSLRLSCAASGSIFSINAMGWYRQ |
| | IGHV3S53 V_{H}H | APGKQRELVAAITSGGSTNYADSVKGRFTISRDNAKNTV |
| | germline | YLQMNSLKPEDTAVYYCNA |
| 8 | Amino acids 1-98 of | EVQLVESGGGLVQPGGSLRLSCAASGFTFSSYAMSWVR |
| | GHV3-23*04 V_{H} | QAPGKGLEWVSAISGSGGSTYYADSVKGRFTISRDNSKN |
| | germline | TLYLQMNSLRAEDTAVYYCAR |
| 9 | CDR1_low diversity | XYXMS |
| | (Kabat) wherein each | |
| | X is independently any | |
| | amino acid except C | |
| 10 | CDR2_low | AIXSGGXTYYADSVKG |
| | diversity(Kabat) | |
| | wherein each X is | |
| | independently any | |
| | amino acid except C | |
| 11 | CDR3_low diversity | XXXXXXXXXXXXXXXFDX |
| | (Kabat) wherein each | |
| | X is independently any | |
| | amino acid except C; | |
| | wherein each X | |
| | beginning with X4 and | |
| | ending with X15 may | |
| | be present or absent | |
| 12 | CDR1_low diversity | GFTFSXYX |
| | (IMGT) wherein each | |
| | X is independently any | |
| | amino acid except C | |
| 13 | CDR2_low diversity | IXSGGXT |
| | (IMGT) wherein each | |
| | X is independently any | |
| | amino acid except C | |
| 14 | CDR3_low diversity | ARXXXXXXXXXXXXXXXFDX |
| | (IMGT) wherein each | |
| | X is independently any | |
| | amino acid except C; | |
| | wherein each X | |
| | beginning with X6 and | |
| | ending with X17 may | |
| | be present or absent | |
| 15 | CDR1_low diversity | GFTFSXYXMS |
| | (AbM) wherein each X | |
| | is independently any | |
| | amino acid except C | |
| 16 | CDR2_low diversity | AIXSGGXTY |
| | (AbM) wherein each X | |
| | is independently any | |
| | amino acid except C | |
| 17 | CDR3_low diversity | XXXXXXXXXXXXXXXFDX |
| | (AbM) wherein each X | |
| | is independently any | |
| | amino acid except C; | |
| | wherein each X | |
| | beginning with X4 and | |
| | ending with X15 may | |
| | be present or absent | |
| 18 | CDR1_low diversity | GFTFSXYX |
| | region (Chothia) | |
| | wherein each X is | |
| | independently any | |
| | amino acid except C; | |
| | wherein X8 is an amino | |
| | acid adjacent to the | |
| | CDR defined by | |
| | Chothia | |
| 19 | CDR2_low diversity | XSGGX |
| | (Chothia) wherein | |
| | each X is | |
| | independently any | |
| | amino acid except C | |
| 20 | CDR3_low diversity | XXXXXXXXXXXXXXXFDX |
| | (Chothia) wherein | |
| | each X is | |
| | independently any | |
| | amino acid except C; | |
| | wherein each X | |
| | beginning with X4 and | |
| | ending with X15 may | |
| | be present or absent | |
| 21 | CDR1_High diversity | XXYXMS |
| | (Kabat) wherein each | |
| | X is independently any | |
| | amino acid except C; | |
| | wherein X1 is an amino | |
| | acid adjacent to the | |
| | CDR | |
| 22 | CDR2_High diversity | XISXXGXXTYYADSVKG |
| | (Kabat) wherein each | |
| | X is independently any | |
| | amino acid except C | |
| 23 | CDR3_High diversity | XXXXXXXXXXXXXXXFDX |
| | (Kabat) wherein each | |
| | X is independently any | |
| | amino acid except C; | |
| | wherein each X | |
| | beginning with X4 and | |
| | ending with X15 may | |
| | be present or absent | |
| 24 | CDR1_High diversity | GFTFXXYAMX |
| | region (IMGT) | |
| | wherein each X is | |
| | independently any | |
| | amino acid except C; | |
| | wherein M9 and XI0 | |
| | are amino acids | |
| | adjacent to the CDR | |
| | defined by IMGT | |
| 25 | CDR2_High diversity | XISXXGXXT |
| | region (IMGT) | |
| | wherein each X is | |
| | independently any | |
| | amino acid except C; | |
| | wherein X1 is an amino | |
| | acid adjacent to the | |
| | CDR defined by IMGT | |
| 26 | CDR3_High diversity | ARXXXXXXXXXXXXXXXFDX |
| | (IMGT) wherein each | |
| | X is independently any | |
| | amino acid except C; | |
| | wherein each X | |
| | beginning with X6 and | |
| | ending with X17 may | |
| | be present or absent | |
| 27 | CDR1_High diversity | GFTFXXYAMX |
| | (AbM) wherein each X | |
| | is independently any | |
| | amino acid except C | |
| 28 | CDR2_High diversity | XISXXGXXTY |
| | (AbM) wherein each X | |
| | is independently any | |
| | amino acid except C | |
| 29 | CDR3_High diversity | XXXXXXXXXXXXXXXFDX |
| | (AbM) wherein each X | |
| | is independently any | |
| | amino acid except C; | |
| | wherein each X | |
| | beginning with X4 and | |
| | ending with X15 may | |
| | be present or absent | |
| 30 | CDR1_High diversity | GFTFXXYAMX |
| | region (Chothia) | |
| | wherein each X is | |
| | independently any | |
| | amino acid except C; | |
| | wherein X8 is an amino | |
| | acid adjacent to the | |
| | CDR; wherein A8, M9, | |
| | and XI0 are amino | |
| | acids adjacent to the | |
| | CDR defined by | |
| | Chothia | |
| 31 | CDR2_High diversity | XISXXGXX |
| | region (Chothia) | |
| | wherein each X is | |
| | independently any | |
| | amino acid except C; | |
| | wherein X1, I2, and X8 | |
| | are amino acids | |
| | adjacent to the CDR | |
| | defined by Chothia | |
| 32 | CDR3_High diversity | XXXXXXXXXXXXXXXFDX |
| | (Chothia) wherein | |
| | each X is | |
| | independently any | |
| | amino acid except C; | |
| | wherein each X | |
| | beginning with X4 and | |
| | ending with X15 may | |
| | be present or absent | |
| 33 | Low diversity human | EVQLVESGGGLVQPGGSLRLSCAASGFTFSXYXMSWYR |
| | human-like V_{H}H | QAPGKQRELVSAIXSGGXTYYADSVKGRFTISRDNSKNT |
| | comprising GHV3- | LYLQMNSLRAEDTAVYYCARXXXXXXXXXXXXXXXF |
| | 23*04 VH with | DXWGQGTLVTVSS |
| | Y37Q44/R45/L47, | |
| | wherein each X is | |
| | independently any | |
| | amino acid except C; | |
| | wherein following | |
| | CAR each X beginning | |
| | with X4 and ending | |
| | with X15 may be | |
| | present or absent | |
| 34 | High diversity human | EVQLLESGGGLVQPGGSLRLSCAASGFTFXXYAMXWVR |
| | human-like V_{H}H | QAPGKQREWVSXISXXGXXTYYADSVKGRFTISRDNSK |
| | comprising GHV3- | NTLYLQMNSLRAEDTAVYYCARXXXXXXXXXXXXXX |
| | 23*04 VH with | XFDXWGQGTLVTVSS |
| | Q44/R45, wherein each | |
| | X is independently any | |
| | amino acid except C; | |
| | wherein following | |
| | CAR each X beginning | |
| | with X4 and ending | |
| | with X15 may be | |
| | present or absent | |
| 35 | GHV3-23*04 VH and | GQGTLVTVSS |
| | IGHV3S53 framework | |
| | 4 | |
| 36 | Alpaca IGHV3S53 | GSIFSINA |
| | germline CDRH1 | |
| 37 | Alpaca IGHV3S53 | ITSGGST |
| | germline CDRH2 | |

### Art Cited In The Examples

1. Kaplon, H. et al. Antibodies to watch in 2020. MAbs 12, e1703531 (2020).
2. Rouet, R., Dudgeon, K., Christie, M., Langley, D. & Christ, D. Fully human VH single domains that rival the stability and cleft recognition of camelid antibodies. J. Biol. Chem. 290, 11905-11917 (2015).
3. To, R. et al. Isolation of monomeric human VHs by a phage selection. J. Biol. Chem. 280, 41395-41403 (2005).
4. Hamers-Casterman, C. et al. Naturally occurring antibodies devoid of light chains. Nature 363, 446-448 (1993).
5. Muyldermans, S. Nanobodies: Natural Single-Domain Antibodies. Annu. Rev. Biochem. 82, 775-797 (2013).
6. Ubah, O. C. et al. Next-generation flexible formats of VNAR domains expand the drug platform's utility and developability. Biochem. Soc. Trans. 46, 1559-1565 (2018).
7. Wesolowski, J. et al. Single domain antibodies: Promising experimental and therapeutic tools in infection and immunity. Med. Microbiol. Immunol. 198, 157-174 (2009).
8. Saerens, D., Ghassabeh, G. H. & Muyldermans, S. Single-domain antibodies as building blocks for novel therapeutics. Curr. Opin. Pharmacol. 8, 600-608 (2008).
9. Vazquez-Lombardi, R. et al. Challenges and opportunities for non-antibody scaffold drugs. Drug Discov. Today 20, U71-1283 (2015).
10. Sarker, S. A. et al. Anti-rotavirus protein reduces stool output in infants with diarrhea: A randomized placebo-controlled trial. Gastroenterology 145, 740-748.e8 (2013).
11. Laursen, N. S. et al. Universal protection against influenza infection by a multidomain antibody to influenza hemagglutinin. Science (80-.). 362, 598-602 (2018).
12. Morrison, C. Nanobody approval gives domain antibodies a boost. Nat. Rev. Drug Discov. 18, 485-487 (2019).
13. Iezzi, M. E., Policastro, L., Werbajh, S., Podhajcer, O. & Canziani, G. A. Single-domain antibodies and the promise of modular targeting in cancer imaging and treatment. Frontiers in Immunology (2018). doi:10.3389/fimmu.2018.00273
14. McMahon, C. et al. Yeast surface display platform for rapid discovery of conformationally selective nanobodies. Nat. Struct. Mol. Biol. 25, 289-296 (2018).
15. Moutel, S. et al. NaLi-H1: A universal synthetic library of humanized nanobodies providing highly functional antibodies and intrabodies. Elife 5, 1-31 (2016).
16. Zimmermann, I. et al. Synthetic single domain antibodies for the conformational trapping of membrane proteins. Elife 7, e34317 (2018).
17. Uchański, T. et al. An improved yeast surface display platform for the screening of nanobody immune libraries. Sci. Rep. 9, 1-12 (2019).
18. Shaheen, H. H. et al. A Dual-Mode Surface Display System for the Maturation and Production of Monoclonal Antibodies in Glyco-Engineered Pichia pastoris. PLoS One 8. e70190 (2013).
19. Alford, R. F. et al. The Rosetta All-Atom Energy Function for Macromolecular Modeling and Design. J. Chem. Theory Comput. 13, 3031-3048 (2017).
20. North, B., Lehmann, A. & Dunbrack, R. L. A new clustering of antibody CDR loop conformations. J. Mol. Biol. 406, 228-256 (2011).
21. Pappas, L. et al. Rapid development of broadly influenza neutralizing antibodies through redundant mutations. Nature 516, 418-422 (2014).
22. Miyazaki, N. et al. Isolation and characterization of antigen-specific alpaca (Lama pacos) VHH antibodies by biopanning followed by high-Throughput sequencing. J. Biochem. 158, 205-215 (2015).
23. Li, X. et al. Comparative analysis of immune repertoires between bactrian Camel's conventional and heavy-chain antibodies. PLoS One 11, 1-15 (2016).
24. Vincke, C. et al. General strategy to humanize a camelid single-domain antibody and identification of a universal humanized nanobody scaffold. J. Biol. Chem. 284, 3273-3284 (2009).
25. Sharpe, A. H. & Pauken, K. E. The diverse functions of the PD1 inhibitory pathway. Nat. Rev. Immunol. 18, 153-167 (2018).
26. Peters, S., Kerr, K. M. & Stahel, R. PD-1 blockade in advanced NSCLC: A focus on pembrolizumab. Cancer Treat. Rev. 62, 39-49 (2018).
27. Francisco, L. M., Sage, P. T. & Sharpe, A. H. The PD-1 pathway in tolerance and autoimmunity. Immunol. Rev. 236, 219-242 (2010).
28. Wilson, I. A. & Stanfield, R. L. Antibody-antigen interactions: new structures and new conformational changes. Curr. Opin. Struct. Biol. 4, 857-867 (1994).
29. Stanfield, R. L. & Wilson, I. A. Protein-peptide interactions. Curr. Opin. Struct. Biol. 5, 103-113 (1995).
30. van Dyck, C. H. Anti-Amyloid-β Monoclonal Antibodies for Alzheimer's Disease: Pitfalls and Promise. Biol. Psychiatry 83, 311-319 (2018).
31. Mujić-Delić, A., De Wit, R. H., Verkaar, F. & Smit, M. J. GPCR-targeting nanobodies: Attractive research tools, diagnostics, and therapeutics. Trends Pharmacol. Sci. 35, 247-255 (2014).
32. Miao, Y. & McCammon, J. A. Mechanism of the G-protein mimetic nanobody binding to a muscarinic G-protein-coupled receptor. Proc. Natl. Acad. Sci. U. S. A. 115, 3036-3041 (2018).
33. Rasmussen, S. G. F. et al. Structure of a nanobody-stabilized active state of the β2adrenoceptor. Nature 469, 175-181 (2011).
34. Wingler, L. M., McMahon, C. Staus, D. P., Lefkowitz, R. J. & Kruse, A. C. Distinctive Activation Mechanism for Angiotensin Receptor Revealed by a Synthetic Nanobody. Cell 176, 479-490.e12 (2019).
35. Ahmadzadeh, V., Farajnia, S., Feizi, M. A. H. & Nejad, R. A. K. Antibody humanization methods for development of therapeutic applications. Monoclon. Antib. Immunodiagn. Immunother. 33, 67-73 (2014).
36. Hwang, W. Y. K., Almagro, J. C., Buss, T. N., Tan, P. & Foote, J. Use of human germline genes in a CDR homology-based approach to antibody humanization. Methods 36, 35-42 (2005).
37. Tan, P. et al. “Superhumanized” Antibodies: Reduction of Immunogenic Potential by Complementarity-Determining Region Grafting with Human Germline Sequences: Application to an Anti-CD28. J. Immunol. 169, 1119-1125 (2002).
38. Mader, A. & Kunert, R. Evaluation of the potency of the Anti-idiotypic antibody Ab2/3H6 mimicking gp41 as an HIV-1 vaccine in a rabbit prime/boost study. PLoS One 7, 1-8 (2012).
39. Yan, J., Li, G., Hu, Y., Ou, W. & Wan, Y. Construction of a synthetic phage-displayed Nanobody library with CDR3 regions randomized by trinucleotide cassettes for diagnostic applications. J. Transl. Med. 12, 1-12 (2014).
40. Dunbar, J. et al. SAbDab: the structural antibody database. Nucleic Acids Res. 42, D1140-6 (2014).
41. Bender, B. J. et al. Protocols for Molecular Modeling with Rosetta3 and RosettaScripts. Biochemistry 55, 4748-4763 (2016).
42. Fleishman, S. J. et al. RosettaScripts: a scripting language interface to the Rosetta macromolecular modeling suite. 6, e20161 (2011).
43. Lo, B. K. C. & Lefranc, M.-P. IMGT, The International ImMunoGeneTics Information System®. Antib. Eng. 33, 27-50 (2004).
44. Leinonen, R., Sugawara, H. & Shumway, M. The sequence read archive. Nucleic Acids Res. 39, 2010-2012 (2011).
45. Masella, A. P., Bartram, A. K., Truszkowski, J. M., Brown, D. G. & Neufeld, J. D. PANDAseq: Paired-end assembler for illumina sequences. BMC Bioinformatics 13, 559; author reply 559-60 (2012).
46. Ye, J., Ma, N., Madden, T. L. & Ostell, J. M. IgBLAST: an immunoglobulin variable domain sequence analysis tool. Nucleic Acids Res. 41, 34-40 (2013).
47. Crooks, G. E. WebLogo: A Sequence Logo Generator. Genome Res. 14, 1188-1190 (2004).
48. Hunter, J. D. Matplotlib: A 2D graphics environment. Comput. Sci. Eng. 9, 99-104 (2007).
49. Lefranc, M. P. IMGT, the international imMunoGeneTics information System. Cold Spring Harb. Protoc. 6, 595-603 (2011).
50. Argyros, R. et al. A Phenylalanine to Serine Substitution within an O-Protein Mannosyltransferase Led to Strong Resistance to PMT-Inhibitors in Pichia pastoris. PLoS One (2013). doi:10.1371/journal.pone.0062229
51. Wasilko, D. & Lee, S. E. TIPS: Titerless Infected-Cells Preservation and Scale-Up. Bioprocess. J. (2006). doi:10.12665/j53.wasilkolee

While the present invention is described herein with reference to illustrated embodiments, it should be understood that the invention is not limited hereto. Those having ordinary skill in the art and access to the teachings herein will recognize additional modifications and embodiments within the scope thereof Therefore, the present invention is limited only by the claims attached herein.

1.-7. (canceled)

8. A human-like heavy chain antibody variable domain (VHH) comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering.

9. The human-like VHH of claim 8, wherein the human VH framework further includes substitution of each of the amino acids at positions 37 and 47 with the amino acid at corresponding positions 37 and 47 of the Camelid VHH framework, wherein the amino acid positions are according to Kabat numbering.

10. The human-like VHH of claim 8, wherein the human VH framework comprises the amino acid sequence of the human VH framework encoded by the IGHV3-23*04 gene in which the amino acids at positions 44 and 45 of the human VH framework are each substituted with the corresponding amino acid at positions 44 and 45 of the Camelid VHH framework encoded by the alpaca IGHV3 S53 gene, wherein the amino acid positions are according to Kabat numbering.

11. The human-like VHH of claim 8, wherein the human VH framework comprises the amino acid sequence of the human VH framework encoded by the IGHV3-23*04 gene and the amino acids at positions 37, 44, 45, and 47 of the human VH framework are each substituted with the corresponding amino acid at positions 37, 44, 45, and 47 of the alpaca VHH framework encoded by the IGHV3S53 gene, wherein the amino acid positions are according to Kabat numbering.

12. The human-like VHH of claim 8, wherein the human-like VHH comprises the amino acid sequence set forth in SEQ ID NO:1, SEQ ID NO: 2, SEQ ID NO: 3, or SEQ ID NO:4.

13. The human-like VHH of claim 8, wherein the human-like VHH is fused at the C-terminus to a polypeptide or peptide that enables the human-like VHH to be displayed on the outer surface of a host cell or a bacteriophage.

14. The human-like VHH of claim 13, wherein the polypeptide is a fragment crystallizable (Fc) region of an immunoglobulin or the coat protein of a bacteriophage and the peptide is a first peptide capable of binding to a second peptide fused to a bacteriophage coat protein that is displayed on the surface of the bacteriophage encoded by a second nucleic acid molecule and which is encoded by a second nucleic acid molecule.

15.-20. (canceled)

21. A display system for displaying a human-like heavy chain antibody variable domain (VHH) on the outer surface of a host cell comprising:
(a) a plurality of first expression vectors, each first expression vector comprising a nucleic acid molecule encoding
(i) a human-like VHH fusion protein comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering, and
(ii) a first Fc polypeptide;
(b) a multiplicity of second expression vectors, each second expression vector comprising a nucleic acid molecule encoding a bait polypeptide comprising a second Fc polypeptide fused to a polypeptide or peptide that enables the second Fc polypeptide to be displayed on the outer surface of a host cell, the first and second Fc polypeptides acting, when the human-like VHH fusion protein is produced in the host cell, to cause the display of the human-like VHH fusion protein via pairwise interaction between the first and second Fc polypeptides; and
(c) host cells for transforming with the plurality of first expression vectors and multiplicity of second expression vectors.

22. The display system of claim 21, wherein the human VH framework further includes substitution of each of the amino acids at positions 37 and 47 with the amino acids at corresponding positions 37 and 47 of the Camelid VHH framework, wherein the amino acid positions are according to Kabat numbering.

23. The display system of claim 21, wherein the human VH framework comprises the amino acid sequence of the human VH framework encoded by the human IGHV3-23*04 gene in which the amino acids at positions 44 and 45 of the human VH framework are each substituted with the corresponding amino acid at positions 44 and 45 of the Camelid VHH framework encoded by the alpaca IGHV3 S53 gene, wherein the amino acid positions are according to Kabat numbering.

24. The display system of claim 21, wherein the human VH framework comprises the amino acid sequence of the human VH framework encoded by the IGHV3-23*04 gene and the amino acids at positions 37, 44, 45, and 47 of the human VH framework are each substituted with the corresponding amino acid at positions 37, 44, 45, and 47 of the Camelid VHH framework encoded by the alpaca IGHV3S53 gene, wherein the amino acid positions are according to Kabat numbering.

25. The display system of claim 21, wherein each human-like VHH comprises the amino acid sequence wherein the human-like VHH comprises the amino acid sequence set forth in SEQ ID NO:1, SEQ ID NO: 2, SEQ ID NO: 3, or SEQ ID NO:4.

26. The display system of claim 21, wherein the host cell is a yeast or filamentous fungus.

27.-32. (canceled)

33. A method for identifying a human-like heavy chain antibody variable domain (VHH) that binds a target of interest, the method comprising:
(a) providing a plurality of transformed host cells comprising
(i) a plurality of first expression vectors, each first expression vector comprising a nucleic acid molecule encoding a human-like VHH fusion protein comprising
(aa) comprising three synthetically generated complementarity determining region (CDR) areas in a human antibody heavy chain variable domain (VH) framework in which the amino acids at each of positions 44 and 45 of the human VH framework are substituted with the amino acids at the corresponding positions of a Camelid heavy chain antibody variable domain (VHH) framework, wherein the amino acid positions are according to Kabat numbering, and
(bb) a first Fc polypeptide; and
(ii) a multiplicity of second expression vectors, each second expression vector comprising a nucleic acid molecule encoding a bait polypeptide comprising a second Fc polypeptide fused to a polypeptide or peptide that enables the second Fc polypeptide to be displayed on the outer surface of a host cell, the first and second Fc polypeptides acting, when the human-like VHH fusion protein is produced in the host cell, to cause the display of the human-like VHH fusion protein via pairwise interaction between the first and second Fc polypeptides;
(b) cultivating the transformed host cells under conditions to induce expression of the human-like VHH fusion proteins and the bait polypeptide to produce induced host cells in which the bait polypeptide is displayed on the outer surface of the transformed host cells and the human-like VHH fusion protein is in a pairwise interaction with the bait polypeptide;
(c) contacting the induced host cells with the target of interest conjugated to a detection moiety; and
(d) detecting the detection moiety and selecting the host cells that express the human-like VHH fusion protein that binds the target of interest.

34. The method of claim 33, wherein the human VH framework further includes substitution of each of the amino acids at positions 37 and 47 with the amino acid at corresponding positions 37 and 47 of the Camelid VHH framework, wherein the amino acid positions are according to Kabat numbering.

35. The method of claim 34, wherein the VH framework comprises the amino acid sequence of the human VH framework encoded by the IGHV3-23*04 gene in which the amino acids at positions 44 and 45 of the human VH framework are each substituted with the corresponding amino acid at positions 44 and 45 of the Camelid VHH framework encoded by the alpaca IGHV3S53 gene, wherein the amino acid positions are according to Kabat numbering.

36. The method of claim 34, wherein the VH framework comprises the amino acid sequence of the human VH framework encoded by the IGHV3-23*04 gene and the amino acids at positions 37, 44, 45, and 47 of the human VH framework are each substituted with the corresponding amino acid at positions 37, 44, 45, and 47 of the Camelid VHH framework encoded by the alpaca IGHV3 S53 gene, wherein the amino acid positions are according to Kabat numbering.

37. The method of claim 34, wherein each human-like VHH comprises the amino acid sequence wherein the human-like VHH comprises the amino acid sequence set forth in SEQ ID NO:1, SEQ ID NO: 2, SEQ ID NO: 3, or SEQ ID NO:4.

38. The method of claim 34, wherein the host cell is a yeast or filamentous fungus.

39.-44. (canceled)
